(19) United States
(12) Patent Application Publication
(10) Pub. No.: US 2006/0162026 A1
(43) Pub. Date: Jul. 20, 2006

(54) Expressing TGF-beta proteins in plant plastids

(76) Inventors: Karen Oishi, St. Louis, MO (US); David Williams, Eureka, MO (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/317,169

(22) Filed: Dec. 23, 2005

Related U.S. Application Data
(60) Provisional application No. 60/639,189, filed on Dec. 23, 2004.

(51) Int. Cl. A01H 1/00; A01H 5/00; C12N 5/04; C12N 15/82; A61K 36/81

(52) U.S. Cl. 800/288; 800/317.3; 435/468; 435/419; 424/751

(57) ABSTRACT

Bioactive TGF-β proteins are expressed in transgenic plastids. The TGF-β proteins are used for therapeutic and diagnostic purposes. In particular, Mullerian Inhibitor Substance (MIS), either full length or truncated proteins, are expressed in plastids and used for treating various cancers that contain MIS receptors such as ovarian cancer, breast cancer and prostate cancer.

### FIELD OF THE INVENTION

The present inventions relates to the expression of bioactive TGF-β proteins in plastids. The TGF-β proteins are purified and used in diagnostic and therapeutic applications. In particular, Mullerian Inhibitor Substance (MIS) is made in plastids, purified and used to treat cancers that possess MIS receptors such as ovarian, breast and prostate cancers.

### BACKGROUND OF THE INVENTION

Proteins of the TGF-.beta. family (TGF-β function to mediate many important embryogenic and immune functions including chemotaxis, production of extracellular matrix, regulation of cell growth and differentiation, and development and regulation of the immune system. Thus, these molecules could be used in a great variety of therapies if available in sufficient quantities. Epithelial ovarian cancers, for example, are the fifth most common malignancy in women. Each year approximately 26,600 new cases of epithelial ovarian cancer are diagnosed, of which 55% die of the disease annually. Studies have shown that Mullerian Inhibiting Substance (MIS) also known as Anti-Mullerian Hormone (AMH), a TGF-β family member, may potentially be an effective therapy for the highly lethal advanced ovarian carcinomas. The current production systems for MIS, including mammalian, bacterial, and nuclear plant transformation systems, however, are not capable of providing MIS at levels required for clinical trials or commercial applications. The bacterial and mammalian systems are incapable of directly producing the biologically active C-terminal MIS without a refolding process. Further, all of these prior systems cannot produce adequate quantities of the holo-MIS precursor, and they suffer from additional disadvantages as well.

Although the biotechnology industry has directed its efforts to eukaryotic hosts like mammalian cell tissue culture, yeast, fungi, insect cells, and transgenic animals, to express recombinant proteins, these hosts may suffer particular disadvantages.

A plant based production of TGF-β proteins including MIS proteins has also been reported in US Published Patent Application 20030033637. That production process is limited to nuclear transformation of plants using plant signal peptide fused with MIS genes. The problem with nuclear transformation is that the holoMIS will contain plant specific N-linked glycans which have been found to be immunogenic. Plastids do not have the process to glycosylate proteins.

Mullerian Inhibiting Substance (MIS) is a well charactereized TGF-β protein. U.S. Pat. No. 5,047,336 discloses DNA sequences, recombinant DNA molecules and processes for producing MIS-like polypeptides. MIS is known to be useful in treating a variety of conditions such as, for example, cancer, fertility, respiratory distress syndrome, and excess androgen states. See, for example, U.S. Pat. No. 5,198,420; U.S. Pat. No. 5,661,126; U.S. Pat. No. 5,912,224; US Published Application 20020031500; and US Published Application 20040151693.

The biochemical, technical, and economic limitations on existing prokaryotic and eukaryotic expression systems have created substantial interest in developing new expression systems for the production of heterologous proteins. To that end, plant plastids represent a suitable alternative to other host systems because of the advantageous economics of growing plant crops, plant suspension cells, and tissues such as callus; the ability to synthesize proteins in leaves, and in storage organs like tubers, seeds, and fruits; the capability of plants for protein bioproduction at very large scales; and the ability to produce the protein in an environment free of human pathogens. Plant-based expression systems may be more cost-effective than other large-scale expression systems for the production of therapeutic proteins.

The present invention contemplates producing a bioactive TGF-β protein, such as the MIS protein, in a plant plastid system. The MIS protein of the present invention may be any full length MIS precursor (e.g., holo-MIS), the 140 kD homodimer from which the bioactive C-terminal homodimer can be released, or the bioactive C-terminal peptide fragment which acts as a inhibitor of ovarian cancer at the desired concentration, and which under pathological conditions, modulates the functional activities of individual cells and tissues.

The MIS of the present invention belongs to the TGF-β superfamily, which includes various TGF-.beta. isoforms, GDF isoforms (Growth/Differentiation Factors), Inhibins, Activins, MIS (Mullerian Inhibiting Substance or Anti-Mullerian Hormone), BMP (bone morphogenetic proteins), dpp (decapentaplegic protein), Vg-1, and MNSF (monoclonal nonspecific suppressor factor). Proteins of this family share common features including sequence similarity, protein structure and post-translational processing, receptor interactions, and biological function as cytokines. The MIS product of the present invention can act as a growth inhibitor of ovarian and other cancer cells originating in the reproductive organs of both males and females, such as breast cancer and prostate cancer.

The plastid-derived MIS protein of the present invention has significant potential to serve as a novel, non-toxic, and highly specific therapeutic agent for tumors of Mullerian origin.

A number of additional observations support the need to search for more efficient means to produce MIS as an anticancer therapeutic. First, MIS activity is cleavage-dependent, and tumor cells in vitro do not have the ability to do this effectively. Thus, clinical applications may require administration of the fully activated form (C-terminal homo-dimer fragment). The apparent half-life of purified carboxyl-terminal MIS may be short in vivo and, thus, larger doses may be required for regression of ovarian tumors. Attempts to express the C-terminal bioactive component directly in either bacterial or mammalian cell systems have been unsuccessful. It is not clear whether appropriate assembly of the C-terminal homo-dimer is dependent on the presence of the N-terminal pro-sequences or if its bioactivity in mammalian cells precludes effective bioproduction. The projected amounts of MIS required for initial in vitro and in vivo antiproliferative studies may be met by current protocols that involve CHO-based bioproduction of the holo-MIS and enzymatic activation in vitro. However, more effective strategies are needed to meet expected requirements for holo-MIS and carboxy-terminal MIS for later clinical trials and potential commercial markets. The method provided by the present invention will meet these needs by yielding more efficient and cost-effective means for producing bioactive therapeutic proteins that mimic the structure and biologically activity of native proteins.

### BRIEF DESCRIPTION OF THE DRAWINGS

FIG. 1 shows amino acid sequences and coding sequences for carboxyl terminal HMIS. (A) The amino acid sequence of the carboxyl terminal bioactive human Mullerian Inhibiting Substance (C-term hMIS) for CTT expression. The N-terminal methionine (bold M) is not present in native carboxyl terminal MIS. (B). Nucleotide sequence of the coding sequence of carboxyl terminal hMIS synthetically produced for generation of CTT transformation expression cassettes.

FIG. 1B shows the sequence for hMIS and two 3′ translation stop codons, and a 5′ flanking EcoRI site and a 3′ flanking XbaI site.

FIG. 2 shows the rbsC-term hMIS Expression Cassette and the 363 nt EcoRI/XbaI DNA fragment.

FIG. 3 shows the pLDpsbAHSA transformation expression vector as disclosed by Millan, et al., (2003), “A chloroplast transgenic approach to hyper-expression and purify Human Serum Albumin, a protein highly susceptible to proteolytic degradation.” Plant Biotechnology Journal, v1:71-79 FIG. 4 shows the pLDrbs C-term HMIS and pLDPpsbA C-term HMIS transformation/expression vector.

FIG. 5 shows the 428 amino acids of N-terminal hMIS.

FIG. 6 shows the 1284 nucleotides of N-terminal hMIS.

FIG. 7 shows the oCTT26 primer and the oCTT27 primer.

FIG. 8 shows a flow diagram for the generation of pLDrbs prohMIS transformation/expression vector.

FIG. 9 shows the oCTT13 primer.

FIG. 10. shows the 1641 nucleotides of EcoRI, rbs-synthetic proMIS, XbaI.

FIG. 11 shows the primers oCTT15, oCTT21 and oCTT22.

FIG. 12 shows a flow diagram for the generation of the 5′ PpsbA prohMIS cassette.

FIG. 13 shows the 1814 nucleotides of EcoRI-XbaI PpsbA prohMIS.

FIG. 14 shows two prohMIS transformation expression cassettes.

FIG. 15 shows the PCR primers used for analysis of transformed chloroplast DNA.

FIG. 16 shows the sequences for primers oCTT36, oCTT37, oCTT14 and oCTT18.

FIG. 17 shows agarose gel electrophoresis results of the analysis of several PCR products.

FIG. 18 is a picture of a Petri dish showing sprouts from a screening of seeds on MS spectinomycin selection medium.

FIG. 19 is a Western blot from the analysis of proMIS from CHO- and rbs prohMIS transplastomic plants. (A) CHO-derived holo MIS is a 170 kDa glycoprotein under non-reducing conditions and 75 kDa glycoprotein under reducing conditions when analyzed on 10% Tris-glycine SDS-PAGE (BioRad, Inc) and detected with rabbit polyclonal antibody to CHO-derived proMIS (noncleaved). (B) Cross-reacting protein bands co-migrating with CHO-derived prohMIS was detected when rabbit polyclonal anti-CHO derived proMIS was used during western analysis. Pro-hMIS was present in the TTEB crude and 40% AS pellet samples.

FIG. 20 shows the results of slot blot Western analysis of C-term hMIS. The first 2 columns are duplicates of C term hMIS Control from plasmin cleaved CHO-derived hMIS, 50, 25, 12, 6, 3, 1.5, 0.75 ng in 10 ug BSA (A-G) and (H) 10 ug BSA. Panal 1—40% AS pellet after dialysis in 25 mM glycine pH 10.5 at (A) 1:5, (B) 1:10 and (C) 1:20 dilution. Panal 2—DEAE Flow through fraction at (D) 1:5, (E) 1:10 and (F) 1:20 dilution. Panal 3—50 uL of 25 mM glycine 100 mM NaCl 10 mL elution fractions (G) #1, (H) #2, (A) #3, (B) #4 and (C) #5 and Panal 4—50 uL of 25 mM glycine 300 mM NaCl 10 mL elution fractions (D) #1, (E) #2, (F) #3 and (G) #4.

FIG. 21 shows pictures of rbsC-term hMIS and PpsbA C-term hMIS biomass generation in tobacco. Plants are propagated in the (A) greenhouse and (B) field in soil with no spectinomycin selection. Both the C-term hMIS expression cassette and protein were detected in these plants.

FIG. 22 is a table showing the elements of preferred transformation/expression vectors.

FIG. 23 shows the coding sequences for His-tagged HoloMIS used in pCTT150 and pCTT151.”

FIG. 24 is a schematic of one of the purification schemes for full length HMIS.

FIG. 25 is a chromatograph of full length HMIS.

FIG. 26 shows the results of a slot blot ELISA.

FIG. 27 is a table showing the results of a Mullerian Duct Regression assay.

FIG. 28 shows the results of a Western blot analysis for full length hMIS expressed in chloroplasts that has been cleaved to generate cMIS.

FIG. 29 is a schematic of a purification process for cMIS from transplastomic plants.

FIG. 30 shows an exclusion chromotagraph in Panel A and the results of a slot blot ELISA in Panel B.

FIG. 31 shows a chromatograph in Panel A and the results of a slot blot ELISA in Panel B.

FIG. 32 shows nucleotide sequences for 5′ UTRs.”

### BRIEF DESCRIPTION OF THE SEQUENCES

SEQ ID NO: 1 is the PCR primer oCTT1 as used according to the subject invention.

SEQ ID NO: 2 is the PCR primer oCTT2 as used according to the subject invention.

SEQ ID NO: 3 is the chloroplast promoter PpsbA flanked with a EcoRI restriction site.

SEQ ID NO: 4 is the Carboxyl Terminal hMIS amino acid sequence as used according to the subject invention.

SEQ ID NO: 5 is the Carboxyl Terminal HMIS coding sequence (C-term HMIS) used according to the subject invention.

SEQ ID NO: 6 is the the sequence for HMIS and two 3′ translation stop codons, and a 5′ flanking EcoRI site and a 3′ flanking XbaI site.

SEQ ID NO: 7 is the rbsC-term HMIS Expression Cassette and the 363 nt EcoRI/XbaI DNA fragment.

SEQ ID NO: 8 is 428 amino acids of N-terminal hMIS.

SEQ ID NO: 9 is 1284 nucleotide of N-terminal hMIS.

SEQ ID NO: 10 is the PCR primer oCTT26 as used according to the subject invention.

SEQ ID NO: 11 is the PCR primer oCTT27 as used according to the subject invention.

SEQ ID NO: 12 the PCR primer oCTT13 as used according to the subject invention.

SEQ ID NO. 13 is the 1641 nucleotides of EcoRI, rbs-synthetic proMIS, XbaI.

SEQ ID NO. 14 the PCR primer oCTT15 as used according to the subject invention.

SEQ ID NO. 15 the PCR primer oCTT21 as used according to the subject invention.

SEQ ID NO. 16 the PCR primer oCTT22 as used according to the subject invention.

SEQ ID NO. 17 is the 1814 nucleotides of EcoRI-XbaI PpsbA prohMIS.

SEQ ID NO. 18 the PCR primer oCTT36 as used according to the subject invention.

SEQ ID NO. 19 the PCR primer oCTT37 as used according to the subject invention.

SEQ ID NO. 20 the PCR primer oCTT38 as used according to the subject invention.

SEQ ID NO. 21 the PCR primer oCTT14 as used according to the subject invention.

SEQ ID NO. 22 the PCR primer oCTT18 as used according to the subject invention.

### SUMMARY OF THE INVENTION

Briefly, in accordance with the present invention, plastids are transformed with an expression cassette containing a polynucleotide sequence that encodes a full length or truncated TGF-β protein wherein the polynucleotide sequence also contains functional regulatory sequences whereby TGF-β protein is expressed in the transgenic plastid and the TGF-β protein thus produced is bioactive, i.e., it binds with its corresponding TGF-β receptor. The TGF-β protein can be the full length protein or a bioactive truncated version of the native TGF-β protein. Modifications can be made to the TGF-β proteins which do not adversely effect its bioactivity. After the TGF-β protein is purified it is formulated into a pharmaceutical composition containing the active TGF-β protein and a pharmaceutically acceptable carrier. Preferably, the pharmaceutical formulation is a sterile solution for parenteral administration, i.e., intramuscular, subcutaneous, or intravenous.

The present invention also relates to plastid transformation vectors and transplastomic plant cells and transplastomic plants, all of which contain a polynucleotide sequence that encodes a full length or truncated TGF-β protein whereby TGF-β protein is expressed in the transgenic plastid/plant and the TGF-β protein thus produced is bioactive, i.e., it binds with its corresponding TGF-β receptor or can be processed to a truncated version that can bind with its receptor. The TGF-β protein can be the full length protein, a bioactive truncated version of the protein, or a modified TGF-β protein. In addition to the polynucleotide expression cassette (coding and regulatory sequences) present in a plastid transformation vector, the vectors will also contain flanking DNA on each side of the polynucleotide expression cassette wherein both flanking sequences are homologous to a DNA sequence of the target plastid genome. The flanking sequences facilitate the stable integration of the expression cassette into the plastid genome through homologous recombination. In a preferred embodiment the plastid transformation vectors contain a selectable marker coding sequence to facilitate the identification of transplastomic plant cells.

The TGF-β proteins produced in plastids according to the present invention can be used in various diagnostic and therapeutic applications. The plastid-produced TGF-β proteins can be used to bind with the corresponding TGF-β receptor. Full length or bioactive truncated TGF-β proteins are administered to mammals in an amount effective to bind with its TGF-β receptor to cause the desired therapeutic and/or diagnostic result.

Of particular interest in the practice of the present invention a plant and/or a plant cell contains transgenic plastids and, in particular embodiments, transgenic chloroplasts, wherein the transgenic plastids contain a heterologous polynucleotide sequence that encodes a full length or bioactive truncated Mullerian Inhibitor Substance (MIS) protein and regulatory sequences functional in the plastid to cause expression of the polynucleotide coding sequence. Preferably, the polynucleotide sequence contains a coding sequence for a selectable marker which is also under control of functional regulatory sequences, to express the selectable marker gene product to facilitate identification and selection of transplastomic plant cells and plantlets in tissue culture. The transgenic plastids are stably integrated with the heterologous polynucleotide and are inherited through organelle replication in daughter cells. The expression cassette for stably transforming plastids to produce MIS comprises exogenous DNA containing a coding sequence for a full length or truncated MIS protein and regulatory sequences functional in plastids to cause expression of the MIS coding sequence.

The present transplastomic MIS producing plants are capable of producing bioactive full length and truncated MIS. The plastid-produced MIS can be used in diagnostic and therapeutic applications. The present invention encompasses methods of treating any cancer that has an MIS receptor associated with it such as ovarian cancer, cervical cancer, breast cancer, and prostate cancer, to name a few. For example, patient having ovarian cancer that produces MIS receptors is treated by administering an effective MIS-binding amount of a full length or truncated MIS protein wherein the full length or truncated MIS protein was produced in a plastid and the full length or truncated plastid produced protein binds with the MIS receptors present in the ovarian cancer and initiates apoptosis of the cancer cells.

### DETAILED DESCRIPTION OF THE INVENTION

The following definitions are applicable when used herein unless specified otherwise.

“C-terminal MIS” (also referred to as C-Term MIS”) refers to the bioactive C-terminal fragment (a homodimer of 11.7 kDa fragments, C-term) of about 25 kDa. While the literature reports the C-terminal fragment as a 25 kDa homodimer the predictive size of the C-terminal fragment is 23.4 kDa and comprised of two monomers of 11.7 kDa size. The C-terminal MIS shows cell-specific antiproliferative activity both in vitro and in vivo, causing regression of the rat Mullerian duct in organ culture and inhibits tumors having MIS receptors such as human endometrial, cervical, prostate, breast, and ovarian cancers.

“Fragments” or “truncations” (truncated proteins) include any portion of an amino acid sequence of an enzyme or polypeptide which retains at least one structural or functional characteristic of the subject post-translational enzyme or polypeptide.

“Fusion proteins” include a protein in which peptide sequences from different proteins are covalently linked together.

“Bioactive TGF-β proteins” include mammalian TGF-β proteins that possess their natural biological activity and include precursor proteins which may possess little or no natural biological activity but which can be processed into the active TGF-β protein species. For example, in the case of MIS, the 140 kDa homodimer has little or no biological activity but can be processed into the active 25 kDa MIS homodimer C terminal fragment. TGF-β proteins are highly conserved, especially in the C-terminal areas such as in MIS. Mammalian TGF-β proteins include but are not limited to primate, porcine, murine and bovine TGF-β proteins. Preferred mammalian TGF-β proteins are human TGF-β proteins. See discussion of molecular weights of glycosylated versus non-glycosylated MIS below under “Mullerian Inhibitor Substance.”

“Holo-MIS” refers to the homodimer MIS precursor protein wherein each monomer comprises both the N-terminal pro-domain (glycosylated portion) and the 11.7 kDa (non-glycosylated) C-terminal region. Holo-MIS serves as the substrate for enzymatic activation which releases the C-terminal fragment as a 25 kD homodimer (non-glycosylated) that functions as the bioactive MIS cytokine. The holo-MIS molecule itself posses little or no biological activity.

Unless specified otherwise “MIS”: refers to a human recombinant MIS protein or peptide and fragments thereof including the full length holo-MIS and C-terminal MIS proteins. In its broadest sense the present invention relates to all mammalian Mulerian Inhibitor Substance proteins including holodimers and C-terminal fragments.

“Mullerian Inhibiting Substance (MIS)” is a protein belonging to the transforming growth factor (TGF)-.beta. (TGF-β) gene family and is produced by the fetal testis as a 140 kDa glycosylated disulfide-linked homodimer that causes regression of the Mullerian duct in the male fetus. Under reducing conditions, the glycoprotein migrates on gel electrophoresis at an apparent molecular weight of 70 kDa. The protein can be proteolytically cleaved by exogenous plasmin into two distinct fragments that migrate electrophoretically under reducing conditions as 57 kDa and 12.5 kDa moieties with cleavage at residue 427 of the intact 535 amino acid monomer. The molecule is also referred to as Anti-Mullarian Hormone (AMH). These molecular weights are for the mammalian derived glycosylated MIS, i.e. 70 kDa for the full length monomer and 140 kDa for the full length dimer. MIS produced in plant plastids will not be glycosylated but will have the same amino acid sequence. The predicted size for the non-glycosylated MIS monomer is 55 kDa and therefore the dimer will be 110 kDa. All references made herein to MIS or C-Term MIS encompass both glycosylated and non-glycosylated MIS proteins.

In practicing the present invention, plastids are transformed with an expression cassette that contains a heterologous polynucleotide sequence that encodes a bioactive TGF-β protein or a bioactive truncated TGF-β protein employing plastid transformation techniques that are well known to one of ordinary skill in the art. Plant cells containing the transformed plastid are then regenerated and grown into a whole plant to produce the desired TGF-β protein. Standard plant breeding techniques are employed to develop a commercial line of TGF-β producing plant lines. The transplastomic plants are harvested and the TGF-β protein is extracted from the plant biomass and purified using known extraction and purification techniques. The purified TGF-β protein is then used in diagnostic, therapeutic, and analytical applications.

The plastid to be transformed can be any plastid or pro-plastid cell. Suitable plastids include chloroplasts (green tissue), chromoplasts (pigmented tissue), etioplasts, and amyloplasts (starch accumulating non-pigmented tissue). Chromoplasts and amyloplasts can dedifferentiate into pro-plastids in culture. By employing the correct regulatory sequences the TGF-β proteins can be expressed in any chloroplast tissues. In a preferred embodiment of the present invention the TGF-β proteins are expressed in chloroplasts. In a particularly preferred embodiment holo-MIS and C-Term MIS are expressed in tobacco chloroplasts.

Plants that can be transformed with the TGF-β expression cassette can be any higher or lower plant, particularly dicots and monocots. Any plant or crop that produces a prolific biomass of tissue where the TGF-β protein is expressed is preferred. A particularly preferred crop is tobacco for its high yield of green tissue and the fact that it is not a food crop. Other suitable crops include corn and soybeans. Chloroplasts from all lower and higher plants are very similar in properties, and the present invention is therefore directed to all such organisms and their chloroplasts.

Any TGF-β protein can be employed in practicing the present invention for expression in a plant plastid. Suitable TGF-β proteins include TGF-β. Isoforms (isoforms 1, 2, 3, 4 and 5), GDF isoforms (Growth/Differentiation Factors), Inhibins, Activins, MIS (Mullerian Inhibiting Substance or Anti-Mullerian Hormone), BMP (bone morphogenetic proteins), dpp (decapentaplegic protein), Vg-1, and MNSF (monoclonal nonspecific suppressor factor). The TGF-β protein can be from any mammalian species such as for example primate, porcine, murine and bovine TGF-β proteins. Preferred mammalian TGF-β proteins are human TGF-β proteins. Preferred human TGF-β proteins include hHolo-MIS and hC-Term MIS.

Plastid transformation is accomplished with a transformation vector. The vector is prepared by flanking the expression cassette with flanking sequences that are homologous to the plastid genome. The two flanking sequences cause insertion of the expression cassette by homologous recombination. Insertion of the expression cassette can therefore be targeted to a specific position within the plastid genome. In a preferred embodiment the expression cassette is inserted into the spacer region between two functional plastid genes. The assembly and use of the vector is done employing routine molecular biology techniques well known to one of ordinary skill in the art. See WO 09910513 titled “Universal chloroplast integration and expression vectors, transformed plants and products thereof” for a description of chloroplast vectors as well as the following U.S. Pat. Nos. 5,932,479; 6,680,426; 6,642,053; 6,004,782; 5,693,507; 5,877,402; 6,388,168; and 5,451,513; all of which are incorporated herein by reference.

The complete DNA sequence of the plastid genome of tobacco has been reported (Shinozaki et al., EMBO J. (1986) 5:2043-2049). Complete DNA sequences of the plastid genomes from liverwort (Ohyama et al., Nature (1986) 322:572-574) and rice (Hiratsuka et al., Mol. Gen. Genet. (1989) 217:185-194), have also been reported.

Where the regions of homology are present in the inverted repeat regions of the plastid genome (known as IRA and IRB), two copies of the transgene are expected per transformed plastid. Where the regions of homology are present outside the inverted repeat regions of the plastid genome, one copy of the transgene is expected per transformed plastid. The regions of homology within the plastid genome are approximately 4 kb in size or more. Smaller regions of homology may also be used such as 1 kb and as little as 100 bp can provide for homologous recombination into the plastid genome. However, the frequency of recombination and thus the frequency of obtaining plants having transformed plastids decreases with decreasing size of the homology regions. Preferred flanking sequences are about 4 kb in size.

In addition to the flanking sequences, the vector will contain the expression cassette that includes the coding region(s) of the genes to be expressed and plastid-functional regulatory sequences. The coding region is the cDNA of the desired TGF-β protein and any other gene of interest that may be present in the expression cassette, such as a selectable marker gene. The coding regions should be modified if necessary to start with an ATG sequence so that the first amino acid of the expressed protein is methionine.

The regulatory sequences can be any regulatory sequences that are functional in a plastid and include promoters, 5′ UTRs, ribsome binding sites, 3′ termination sequences, and the like. Suitable plastid promoters include, for example, promoters associated with the following plastid genes: 16S ribosomal RNA operon (Prrn), psbA gene (PsbA), the atpB gene or the rbcL gene (PrbcL). Bacterial promoters often function in plastids and can be used, however, plastid promoters are preferred. A preferred plastid promoter is the Prrn promoter.

In an alternative embodiment, chloroplast promoters that express the gene product in non-green tissues can be used to express a selectable marker gene so that non-green tissue can be used as a transformation target and selection can begin in culture in non-green tissue. This allows for more flexibility in the regeneration of transformed cells into plantlets, in particular where it is advantageous or necessary to form somatic embryos in order to regenerate a transgenic plant from a transgenic plant cell. (See WO 2004/005480 titled “Chloroplast Genetic Engineering Via Somatic Embryogenesis”)

3′ Termination sequences can be derived from the same genes from which the promoters are derived. A preferred 3′ termination sequence is the psbA 3′ termination sequence.

Ribosome binding sites can also be present in the expression cassette to enhance expression of the TGF-β protein gene. The ribosome binding site (RBS) is ideally inserted just downstream from the promoter. Suitable ribosome binding sites include bacteriophage T7 gene 10 leader and the rbcL RBS. See U.S. Pat. No. 6,812,379. A preferred RBS is ggaggcaacc.

As a practical matter, the expression cassette will also have a coding sequence for a selectable marker protein. The selectable marker gene can encode, for example, any enzyme that will inactivate a selection agent. Selectable marker genes and selection agents are well known to one of ordinary skill in the art. The selection agent can be a compound that is toxic to the target plant tissue that is being transformed or alternatively it can be a compound that is only toxic to plastids. Selection agents toxic to plants that are known in the art include chloramphenicol, kanamycin, neomycin, basta, glyphosate, etc., as well as their corresponding selectable marker genes, i.e., CAT, bar/PAT, NPTII, EPSPS, etc. Spectinomycin and streptomycin are two selection agents that are only toxic to the plastids. Expression of the aadA gene confers resistance to spectinomycin and streptomycin, and thus allows for the visual identification of plant cells expressing this marker. The aadA gene product allows for continued growth and greening of cells whose chloroplasts comprise the selectable marker gene product. Cells which do not contain the selectable marker gene product (non-transformed cells) are bleached. Selection for the aadA gene marker is thus based on identification of plant cells which are not bleached by the presence of streptomycin, or more preferably spectinomycin, in the plant growth medium.

When foreign genes are inserted into the chloroplast genome, it is possible that some of the chloroplast genomes have foreign genes integrated, while others remain as the wild type, which is referred to as heteroplasmy. Therefore, to ensure that only transformed genome exists in cells of transgenic plants, which is referred to as homoplasmy, the selection process is continued through one or more re-culturing phases. To confirm that the wild type genome does not exist at the end of the selection cycle, total DNA from transgenic plants should be probed with the chloroplast border (flanking) sequences (the trnl-trnA fragment if the pLD vector is used). If wild type genomes are present (heteroplasmy), the native fragment size is observed along with transformed genomes. Presence of a large fragment (due to insertion of foreign genes within the flanking sequences) and absence of the native small fragment confirms homoplasmy (Daniell et al, (1998), Containment of herbicide resistance through genetic engineering of the chloroplast genome. Nature Biotechnology 16:345-348).

An alternative to antibiotic or herbicide selection agents are non-antibiotic compounds such as aldehyde dehydrogenases such as betaine aldehyde, acetaldehyde, formaldehyde, proprionaldehyde, and butyraldehyde. The respective selectable marker genes that inactivate these non-antibiotic selection agents are beataine aldehyde dehydrogenase (BADH), acetaldehyde dehydrogenase, formaldehyde dehydrogenase, proprionaldehyde dehydrogenase, and butyraldehyde dehydrogenase. A preferred non-antibiotic selection agent is betaine aldehyde and the preferred selectable marker gene to detoxify betaine aldehyse is BADH.

The vectors employed in the present invention are capable of plastid transformation, particularly of chloroplast transformation. Such vectors would include chloroplast expression vectors such as pLD, pUC, pBR322, pBlueScript, pGEM, and all others identified by Daniell in U.S. Pat. No. 5,693,507 and U.S. Pat. No. 5,932,479. Included are also vectors whose flanking sequences are located outside the inverted repeat of the chloroplast genome. A preferred vector is the pLD vector. A pLDpsbAHSA transformation expression vector is described by Millan, AF, Mingo-Castel, A, Miller M and Daniell H (2003), “A chloroplast transgenic approach to hyper-expression and purify Human Serum Albumin, a protein highly susceptible to proteolytic degradation” Plant Biotechnology Journal, 1:71-79. The vector in Millan et al. is treated with appropriate restriction enzymes so that the HSA coding region can be swapped out with a desired TGF-β coding region. In a preferred embodiment, hMIS or hC-Term MIS coding regions are swapped out to make a pLD hMIS vector or an hC-Term MIS vector. The pLD vector was developed for chloroplast transformation. It contains the 16S rRNA promoter (Prrn) driving the selectable marker gene aadA (aminoglycoside adenyl transferase conferring resistance to spectinomycin) followed by the psbA 3′ region (the terminator from a gene coding for photosystem II reaction center components) from the tobacco chloroplast genome. The pLD vector is a universal chloroplast expression/integration vector and can be used to transform chloroplast genomes of several other plant species because these flanking sequences are highly conserved among higher plants. The universal vector uses tma and tml genes (chloroplast transfer RNAs coding for Alanine and Isoleucine) from the inverted repeat region of the tobacco chloroplast genome as flanking sequences for homologous recombination. Because the universal vector integrates foreign genes within the Inverted Repeat region of the chloroplast genome, it should double the copy number of the transgene (from 5000 to 10,000 copies per cell in tobacco). Furthermore, it has been demonstrated that homoplasmy is achieved even in the first round of selection in tobacco probably because of the presence of a chloroplast origin of replication within the flanking sequence in the universal vector thereby providing more templates for integration.

In developing the constructs, the various fragments comprising the regulatory regions and open reading frame may be subjected to different processing conditions, such as ligation, restriction enzyme digestion, PCR, in vitro mutagenesis, linkers and adapters addition, and the like. Thus, nucleotide transitions, transversions, insertions, deletions, or the like, may be performed on the DNA which is employed in the regulatory regions or the DNA sequences of interest for expression in the plastids. Methods for restriction digests, Klenow blunt end treatments, ligations, and the like are well known to those in the art and are described, for example, by Maniatis et al. (in Molecular Cloning: A Laboratory Manual (1989) Cold Spring Harbor Laboratory Press, Cold Spring Harbor, N.Y.).

Any transformation method may be used to insert the present vector into the plant cells in order to transform the plastids. Electroporation of isolated etioplasts can used as described in the above mentioned patents, but preferably the vectors are introduced into the plants cells by biolistics or gene gun techniques employing microparticles coated with the vector. Preferred miocroparticles include tungsten and gold beads. Whisker transformation is another transformation technique that can be used where small silicon carbide fibers (whiskers) are agitated in a solution containing the vector and plant cells. See Klein et al. (1987) Nature 327:70; and U.S. Pat. Nos. 4,945,050; 5,036,006; 5,100,792; 5,179,022; 5,204,253; 5,371,015; 5,478,744; 5,302,523; and 5,464,765.

After the transformation process is conducted the target plant cells are then cultured, preferably under selection according to the selectable marker gene employed and plants are regenerated. These techniques are routine and well known to one of ordinary skill in the art. When the plant matures then the tissue wherein the TGF-β gene is expressed is harvested and the TGF-β protein is recovered therefrom using routine extraction and purification techniques. For example, chloroplasts can be isolated from crude homogenate of leaves by centrifugation (1500×g) which eliminates most of the cellular organelles and proteins. The chloroplasts can then be burst open by osmotic shock by re-suspending them in a hypotonic buffer. Thereafter several methods are available for further purification including protein precipitation and affinity chromotography.

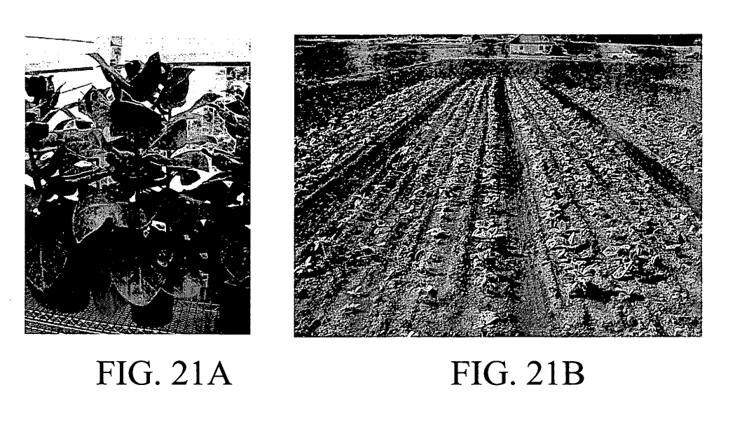

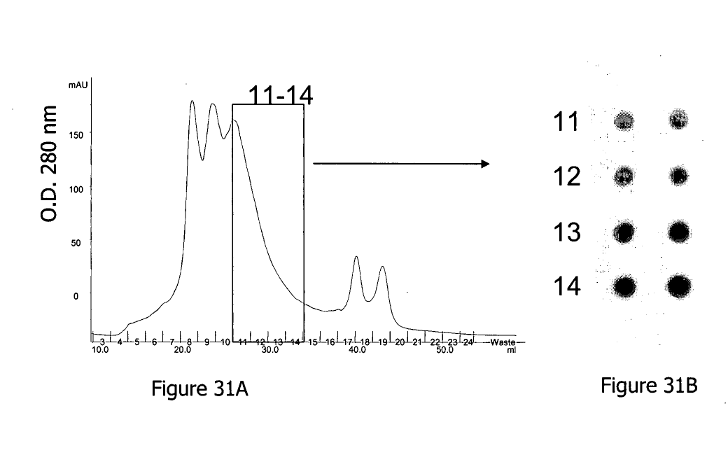

In a preferred embodiment of the present invention transformed chloroplast plant lines are developed employing routine plastid transformation protocols wherein the transplastomic plants produce assembled properly processed MIS protein that can be purified from the plant tissue. Suitable plastid expression vectors preferably include 5′ UTR translational regulators with and without promoters linked to the MIS cDNA (FIG. 22). In addition, the 5′ translational regulator UTR can be developmentally and/or light regulated like the psbA or rbcL or constitutively regulated like the ribosome binding site (rbs) or T7 phage gene 10 (see FIG. 32).

To enhance the ease of purification of hMIS from plant biomass nucleotide sequences for affinity tagged peptides are added 5′ to the HMIS coding sequence whereby a fusion protein is formed having the affinity tagged peptide on the 5′ end. Preferred affinity tagged peptides include the His-tag (Studier F W et al. 1990, Methods Enzymology 185:60-89). The His-tag system has been successfully used to purify recombinant proteins from plants, animals and microbial systems (Novagen. 2002-2003. Protein Expression: Prokaryotic Expression: pETBlue and pET System Overview. Novagen 2002-2003 Catalog. p 84-91. <http://www.novagen.com/SharedImages/Novagen/05_PROEXP.pd>). As shown in FIG. 22 a His-tag was fused to the N-terminal of hMIS cDNA. In pCTT150 and pCTT151 an endoprotease site, like the enterokinase cleavage site, was not placed N-terminal to the HMIS. If desired the fusion proteins can incorporate the enterokinase cleavage site (N-terminal to the hMIS) to assist in the removal of the His-tag from the final purified product.

The TGF-β proteins can be used as is in their full length form or as their processed carboxy terminal active fragment. In the case of MIS this would be the C-Term MIS. The TGF-β proteins can also be pegylated or bound to polyethylene glycol using known methods. The pegylated-TGF-β proteins will be more stable in vivo and have a resulting longer half-life in the body when administered to a mammal in need of treatment. Pegylated pharmaceuticals also many times have lower toxicity profiles and reduced allergenicity. They also are more water soluble. Pegylation of pharmaceuticals including the pegylation of peptides is well known and their preparation is described by Kozlowski et al, BioDrugs 2001, 15 (7) 419-429.

Generally, the pharmaceutical compositions of the present invention may be formulated and administered using methods similar to those used for other pharmaceutically important polypeptides. The polypeptides may be stored in lyophilized form, reconstituted with sterile water just prior to administration and administered intravenously. Preferably, the pharmaceutical formulation of the present TGF-β proteins will be administered in dosages that are determined by routine dose titration experiments for the particular condition to be treated.

In the case of MIS the dosages and the modes of administration will vary and are similar to those that have been used for MIS protein as disclosed in U.S. Pat. No. 4,510,131, the disclosure of which is hereby incorporated by reference. For MIS the dose can be from about 1 to about 1,000 ng/kg or more and usually about 10 to 500 ng/kg. Of course, any pegylated MIS product would have the dosage adjusted to deliver the correct amount of active protein, i.e., holo-MIS or C-Term MIS.

The MIS containing formulations may be administered to the patient in any pharmaceutically acceptable dosage form, including those which may be administered to a patient intravenously as bolus or by continuous infusion over a period of minutes, hours, days, weeks or months, intramuscularly, subcutaneously, intracutaneously, intra-articularly, intrasynovially, intrathecally, periostally, or by oral, topical, or inhalation routes. The MIS can also be impregnated into slow releasing polymer and administered as a solid material such as into a tumor or body tissue/organ. See U.S. Pat. No. 6,692,738. MIS dimers may also be administered intratumorally, peritumorally, intralesionally, or periolesionally to exert local as well as systemic therapeutic effects.

The most effective mode of administration and dosage regimen of MIS dimers will depend upon the type of disease to be treated, the severity and course of that disease, previous therapy, the patient's health status and response to the MIS, and the judgment of the treating physician. MIS dimers may be administered to the patient at one time or over a series of treatments until the desired suppression of a cancer that contains MIS receptors.

The MIS dimers of this invention are useful alone or in combination with other active ingredients such as chemotherapy agents and immune stimulating agents. For example MIS can be combined with interferon and used for treating cancer. See US Published Patent Application 2004/0151693.

The present TGF-β proteins are useful to treat any disease state in a mammal where binding with the TGF-β receptor is desirable. The method of the present invention comprises administering to the mammal an effective amount of a full length or truncated TGF-β protein that has been made in a plant plastid wherein the full length or truncated TGF-β protein binds to its corresponding TGF-β receptor. In the case of MIS, either holo-MIS, C-Term MIS, or pegylated versions of either, is administered to a mammal to treat, for example, a cancer wherein the cancer contains MIS receptors.

The use of MIS in various disease states, primarily cancer, respiratory distress syndrome, and fertility/contraception, is known. Any cancer containing MIS receptors is treatable with MIS. These cancers include vulvar carcinoma, some ocular melanomas, ovarian epithelial cancer, prostate cancer, and breast cancer. See Lane and Donahoe, 1998, Journal of Endocrinology, 158, p. 1-6 and the following US Patents/Published Applications U.S. Pat. Nos. 5,011,687; 5,047,336; 5,198,420; 5,427,780; 5,661,126; 5,912,224; 6,673,352; and 2003/0124620.

The following examples illustrate the practice of the present invention but should not be construed as limiting its scope.

### EXAMPLE 1

### Chloroplast Transformation Vector Construction

A. Construction of Carboxyl-terminal MIS cDNA:

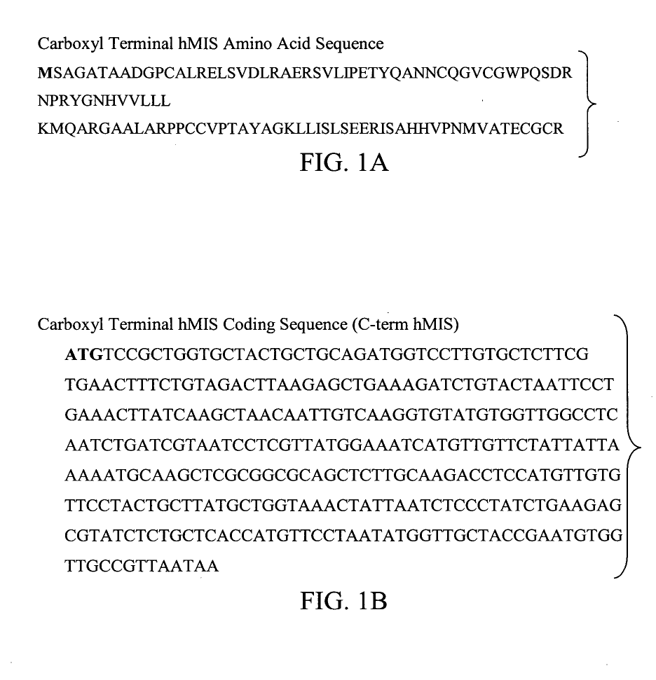

Based on the amino acid sequence of human MIS (GeneBank Accession # P03971) and our knowledge of chloroplast preferred codons, the carboxyl-terminal bioactive MIS DNA fragment was synthesized by BlueHeron Biotechnology, WA. The synthetic gene included an AUG start codon that is required for expression using chloroplast transformation technology (CTT), two 3′ translation stop codons, and a 5′ flanking EcoRI site and a 3′ flanking XbaI site for ease of subsequent cloning. See FIG. 1.

B. Construction of C-terminal MIS Plastid Transformation Expression Vectors:

Two different 5′ regulatory sequences, a psbA gene promoter region and a Shine-Dalgamo ribosome binding site (rbs), were used to express the C-terminal MIS protein (C-term MIS). The Shine-Dalgarno sequence, 5′-AGGAGG-3′, is usually located 10 bp upstream of the AUG start codon and has been shown to be required for ribosome binding to mRNA during translation (Lewin, B (1997) Genes VI).

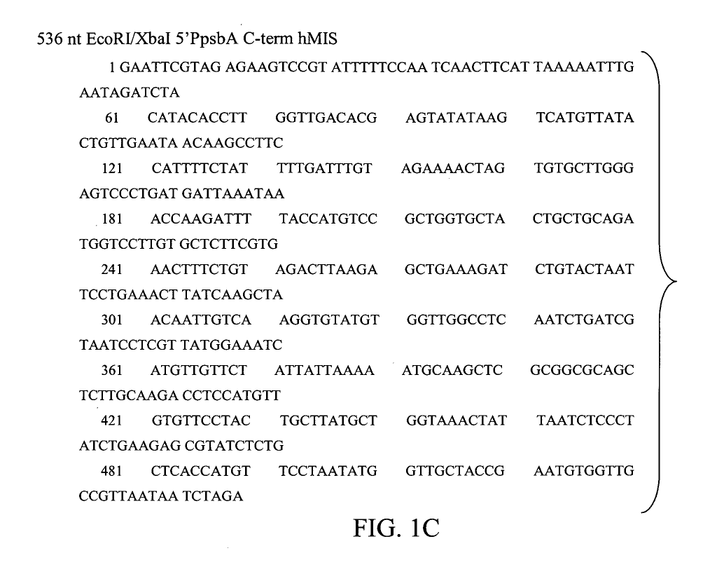

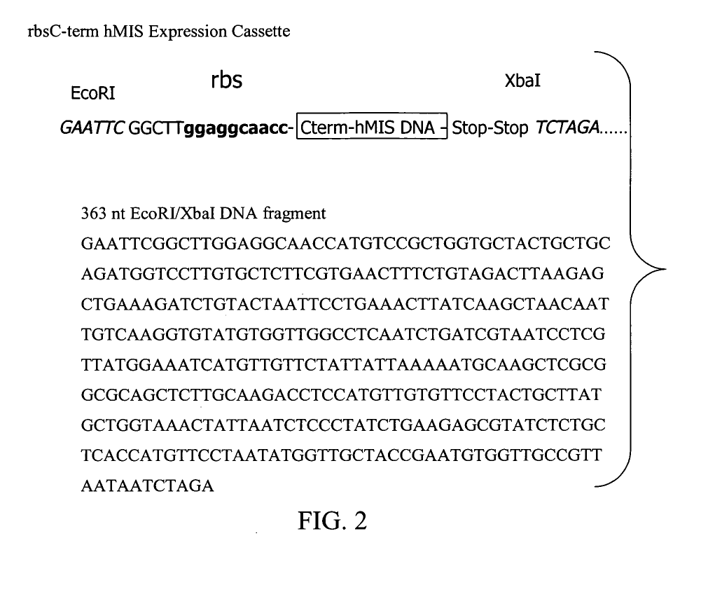

The rbs sequence was placed 5′ of the C-term MIS DNA clone using PCR amplification of the C-term MIS DNA plasmid clone from Blue Heron Biotechnology with the following primers: oCTT1 (5′ GGAGGCAACCATGTCCGCTGGTGCTACTGC3′) (SEQ ID NO: 1) and oCTT2 (5′ GAATTCGGCTTGGAGGCAACCATGTCCG 3′) (SEQ ID NO: 2) forward primers, and the pUC reverse −48 primer (NewEngland Biolabs). The resulting PCR amplified DNA fragment (FIG. 2) was digested with the restriction enzymes EcoRI and XbaI according to the manufacturer's recommendation. The pLDrbsC-term MIS transformation/expression vector was generated by ligation with T4 ligase (according to the manufacturer's recommendation) of the EcoRI, XbaI digested pLDpsbAHSA transformation expression vector (shown in FIG. 3) with rbsC-term MIS EcoRI, XbaI digested PCR fragment. The resulting transformation expression vector will have the 5′psbAHSA fragment replaced by the rbsC-term MIS EcoRI, XbaI DNA.

The chloroplast psbA promoter (PpsbA) flanked with a EcoRI restriction site (5′GAATTCGTAGAGAAGTCCGTATTTTTCCAATCAACTTCATTAAAAATTTGA ATAGATCTACATACACCTTGGTTGACACGAGTATATAAGTCATGTTATACTGT TGAATAACAAGCCTTCCATTTTCTATTTTGATTTGTAGAAAACTAGTGTGCTT GGGAGTCCCTGATGATTAAATAAACCAAGATTTTACC 3′) (SEQ ID NO: 3) was placed 5′ to the C-term MIS DNA (FIG. 1B) using PCR amplification, primer alignment and primer extension.

The pLD PpsbA C-term MIS transformation/expression cassette was generated by ligation with T4 ligase (according to the manufacturer's recommendation) of the EcoRI, XbaI digested pLDpsbAHSA transformation expression vector (FIG. 3) with 5′ PpsbA C-term MIS EcoRI, XbaI digested fragment. The resulting transformation expression vector will replace the 5 psbAHSA fragment with the 5′PpsbA C-term MIS. FIG. 4 shows the pLDrbs C-term MIS and pLDPpsbA C-term MIS Transformation/Expression Vectors.

C. Construction of pro-MIS Plastid Transformation Expression Vectors:

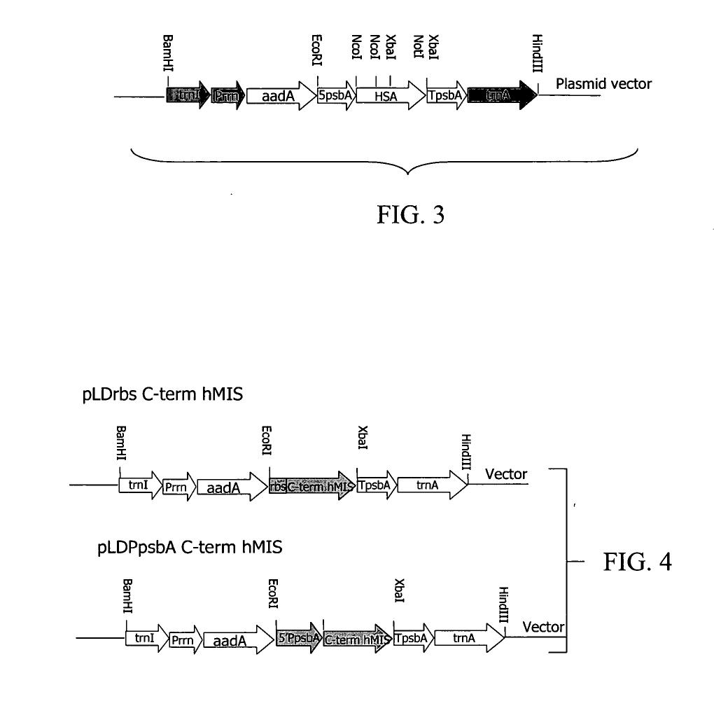

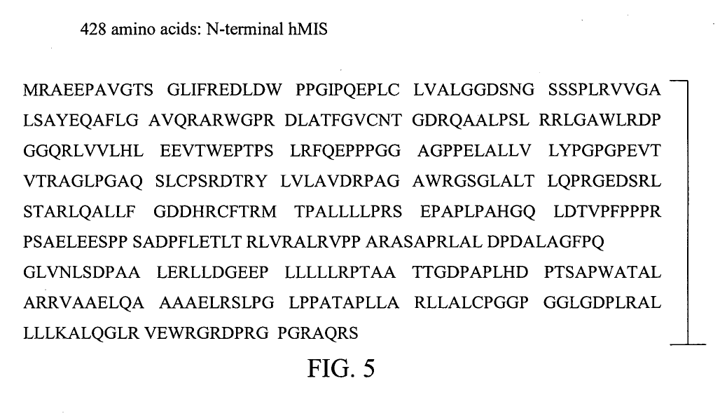

The N-terminal pro-MIS coding sequence was synthesized by BlueHeron Biotechnology, Inc, WA, based on the 428 amino acid nucleotide shown in FIG. 5 and the DNA sequence shown in FIG. 6. Amino acid #2 to #428 represents native MIS amino acid sequence (GeneBank Accession # P03971)

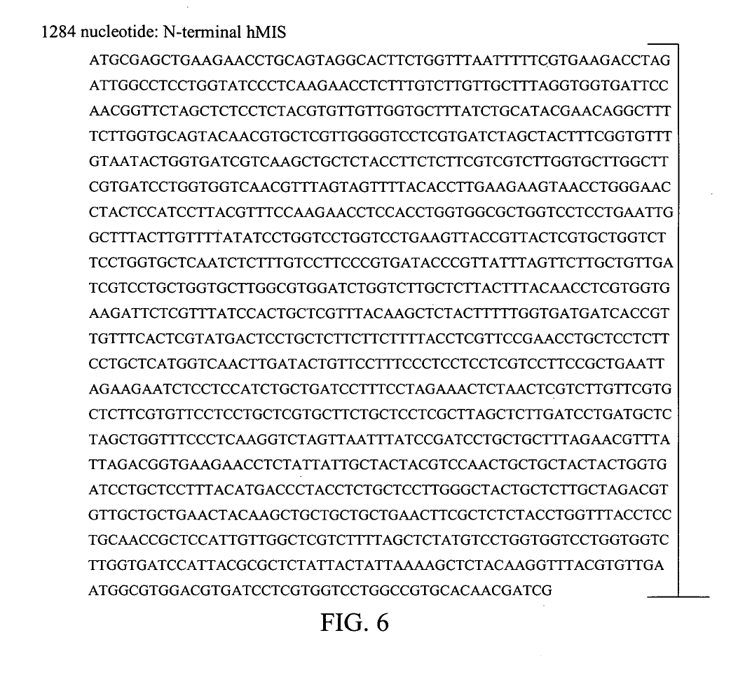

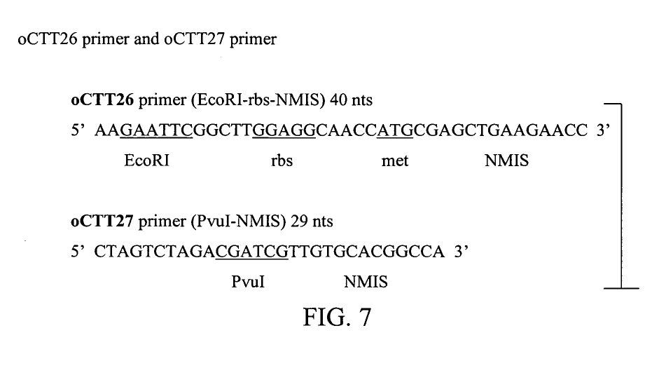

To generate the 5′ rbs pro-MIS coding sequence, the C-term MIS was fused to the N-term pro-MIS (FIG. 3). The rbs 5′ regulatory sequence and a 3′ PvuI site were generated flanking the N-terminal pro-MIS by using forward oCTT26 primer and reverse primer oCTT27 shown in FIG. 7. According to the manufacturer's recommendation the 1.2 kb PCR product was generated with Deep Vent Thermostable DNA Polymerase (NewEngland Biolabs), digested with PvuI and purified according to the manufacturer's recommendation for Wizard PCR purification Kit (Promega, Inc.). See FIG. 8 for a schematic diagram showing the generation of the pLDrbs proMIS Transformation/Expression Vector.

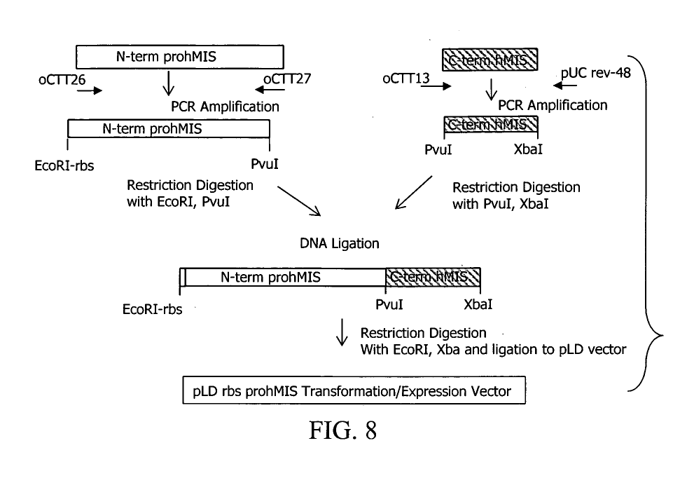

The PvuI site was generated at the 5′ end of the C-term MIS coding sequence by PCR amplification using oCTT13 (FIG. 9), pUC reverse −48 (New England BioLabs) and synthetic C-term MIS clone (FIG. 2).

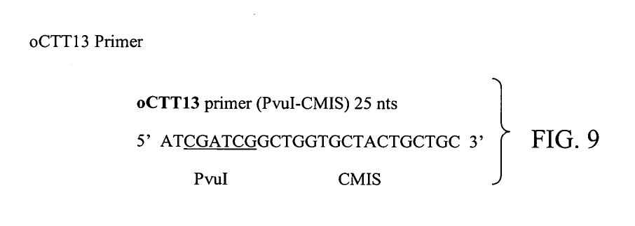

The 0.3 kb PCR DNA fragment was digested with PvuI and purified according to the manufacturer's recommendation for Wizard PCR purification Kit (Promega, Inc). The PvuI digested rbs-Nterminal MIS was ligated to the PvuI digested C-term MIS with T4 DNA ligase, and subsequently purified by gel electrophoresis and Wizard Kit (Promega, Inc.). The resulting 1.5 kb rbs-MIS was digested with EcoRI and XbaI and ligated into LIT28i plasmid vector (New England BioLabs). The DNA sequence of the EcoRI, XbaI DNA fragment is shown in FIG. 10. The EcoRI,XbaI 1.5 kb rbs-MIS fragment was ligated to EcoRI/XbaI digested pLD transformation expression vector.

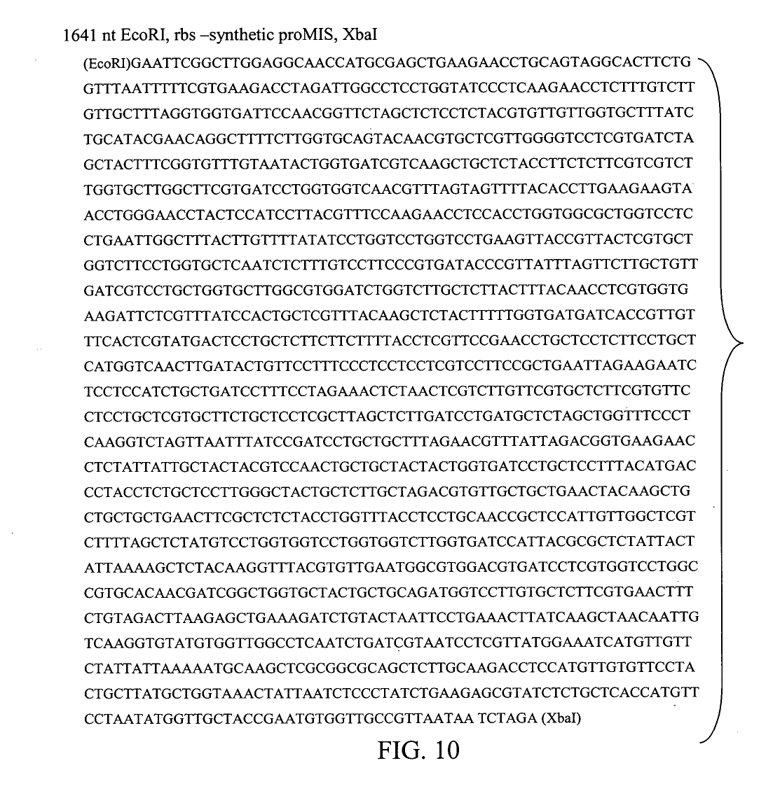

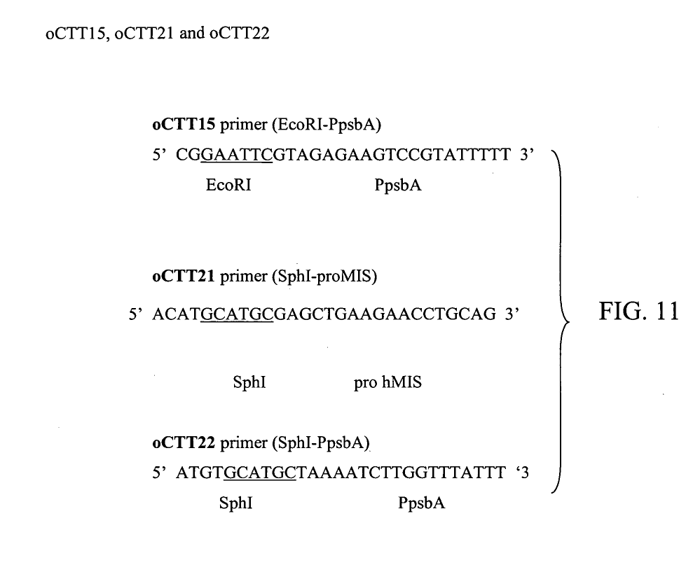

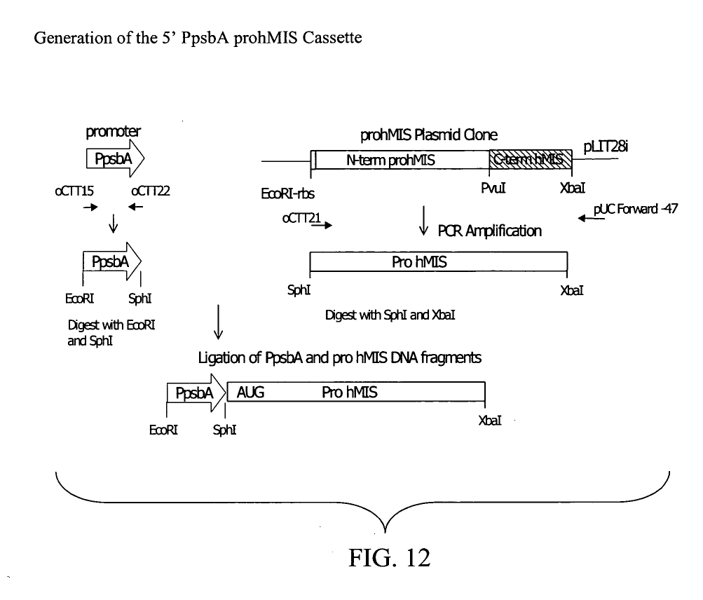

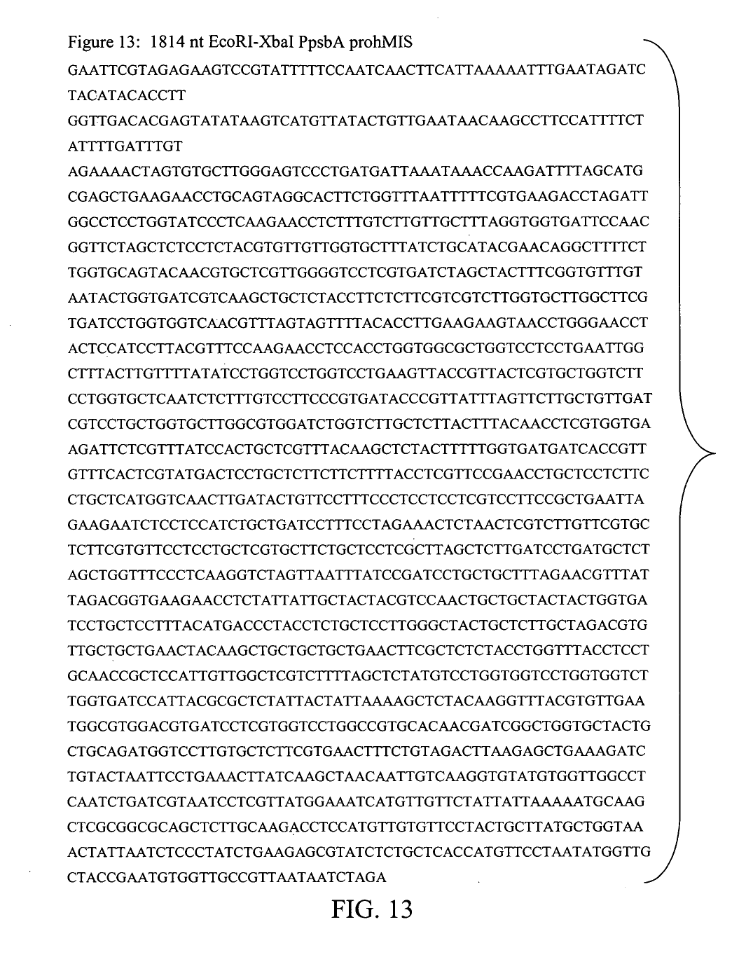

To generate the 5′PpsbA MIS transformation expression vector, a SphI site containing the start codon AUG was generated at the 3′ end of the PpsbA regulatory sequence and at the 5′ end of the proMIS sequence. The pro MIS coding sequence with a 5′ SphI site was generated by PCR amplification using the oCTT21 primer (FIG. 11) and pUC Forward −47 (New England BioLabs) and the rbs pro MIS plasmid clone. The 1.5 kb PCR fragment was digested with SphI and purified using Wizard Kit (Promega, Inc) according to the manufacturer's recommendation. Chloroplast psbA promoter containing a flanking EcoRI and SphI sites was generated by PCR amplification using oCTT15 (FIG. 11) and oCTT22 (FIG. 11). The 200 bp PCR product was digested with SphI and purified using Wizard Kit (Promega, Inc). The SphI digested PpsbA and pro MIS were ligated with T4 DNA ligase. The resulting DNA fragment was purified by agarose gel electrophoresis and the 1.7 kb DNA fragment isolated by Wizard Kit (Promega, Inc.). The 1.7 kb DNA fragment was digested with EcoRI and XbaI and ligated to LIT28i to generate the pLD PpsbA MIS plasmid vector. A schematic diagram showing the generation of the 5′ PpsbA MIS Cassette is shown in FIG. 12. The nucleotide sequence of the EcoRI-XbaI PsbA MIS fragment was determined (SeqWright, Houston Tex.) as shown in FIG. 13. The 1.7 kb EcoRI-XbaI PpsbA MIS DNA fragment was ligated to the pLD transformation vector to generate the pLD PpsbA MIS transformation expression vector. A diagram of the pLD PpsbA MIS and pLDrbs proMIS transformation vectors are shown in FIG. 14.

### EXAMPLE 2

### Plant Transformation

Tobacco (Nicotiana tabacum) plants grown aseptically by germination of seeds on MSO medium containing MS salts were used for transformation. As shown below, for each bombardment a whole leaf was placed abaxial side up on a Whatman filter paper on RMOP medium. DNA from the pLD MIS transformation expression plasmid clones was isolated using Qiagen Plasmid DNA Isolation Kit according to the manufacturer's recommendation (Qiagen, Inc). Gold (0.6 μm) microprojectiles were coated with greater than 900 ug/mL concentration of plasmid DNA containing the transgene. The bombardments and transformation were carried out with the biolistic device PDS 1000/He (Bio-Rad) as described by Daniell H (1997) “Transformation and foreign gene expression in plants mediated by microprojectile bombardment” Meth. Mol. Biol. 62: 453-488. The bombarded tissue was placed on RMOP media for 36-48 hr prior to dissection into 5 mm squares which were then selected on 500 ug/ml spectinomycin media in continuous light at 25° C. To select for homoplasmic plants with 100% transformed chloroplasts, the first true leaf from a regenerated transformed plant was dissected into 2 mm squares for a second round of selection on spectinomycin. After the 1^{st} round of selection usually only a subset of chloroplasts were transformed with the transgene. By the second round of selection substantially all chloroplasts were transformed as confirmed by PCR analysis.

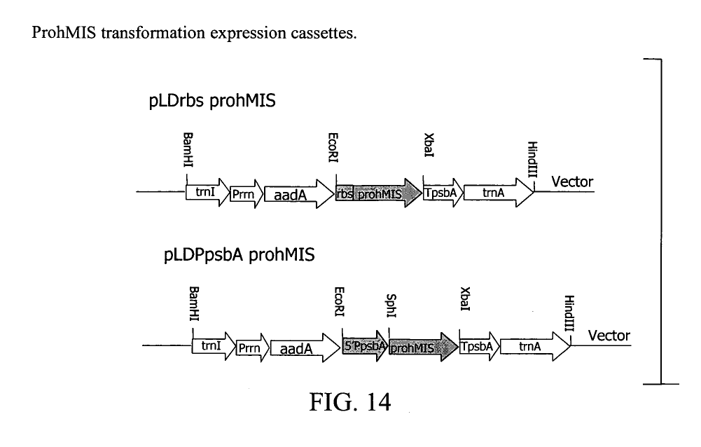

PCR analysis was used to establish the integration of the different MIS expression cassettes in the transformed plants (De Cosa B, Moar W, Lee S B, Millar M, Daniell H (2001) “Overexpression of the Bt cry2Aa2 operon in chloroplast leads to formation of insecticidal crystals” Nat Biotechnol. 19:71-74). Genomic DNA was isolated from plant tissue and used for PCR analysis by REDExtract-N-Amp Plant PCR Kit (Sigma Aldrich) according to the manufacturer's recommendation. The PCR amplification condition (95° C. for 3 min, repeat 30 times at 95° C. for 1 min-65° C. for 45 sec-72° C. for 1 min 30 sec, 72° C. for 3 min followed by 15° C. on hold) is optimum for PCR products 2 kb and smaller. FIG. 15 shows PCR primers that were used for analysis of transformed chloroplast DNA

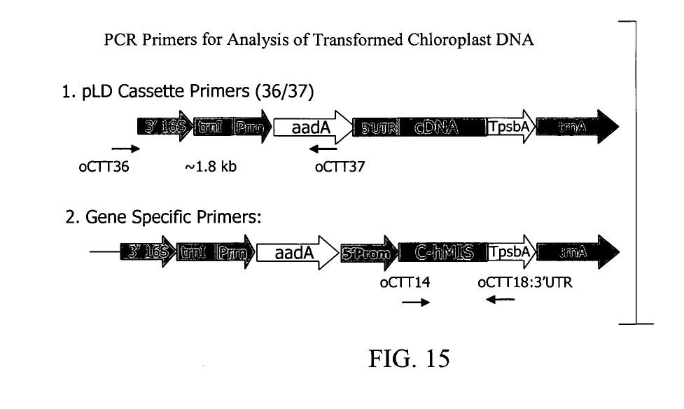

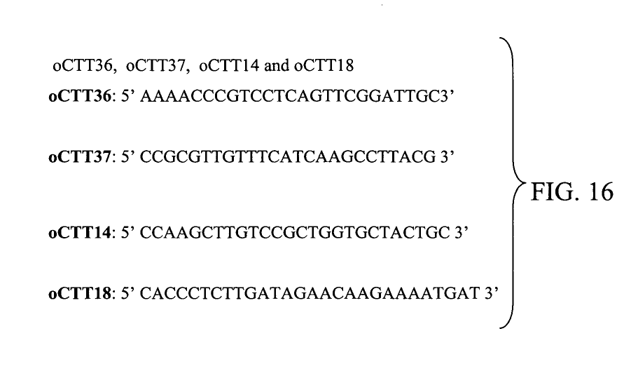

To determine if the transformation expression cassette DNA was integrated into the chloroplast genome, PCR primer oCTT36: 5′ AAAACCCGTCCTCAGTTCGGATTGC3′ (FIG. 16) (SEQ ID NO. 19) which is homologous to a region flanking the site of integration and PCR primer oCTT37: 5′ CCGCGTTGTTTCATCAAGCCTTACG 3′ (FIG. 16) (SEQ ID NO. 20) which is homologous to a region in the aadA gene were used to amplify genomic DNA from non-transformed, (lane 1), transformed C-term MIS (lanes 2 to 12), no DNA (lane 13) and transformed pLD5 psbAHSA (DeCosa et al 2001; lanes 14, 15) plants (FIG. 17A). To determine if the C-term MIS coding sequence was integrated into the chloroplast genome PCR primers homologus to the C-term MIS coding sequence oCTT14: 5′ CCAAGCTTGTCCGCTGGTGCTACTGC 3′ (FIG. 16) (SEQ ID NO. 21) and homologous 3′ TpsbA UTR region, oCTT18: 5′ CACCCTCTTGATAGAACAAGAAAATGAT 3′ (FIG. 16) (SEQ ID NO. 22) were use to PCR amplify genomic DNA from non-transformed, C-term MIS transformed and HSA transformed plants (FIG. 17B). Nine of eleven transformed C-term MIS lines had a 1.8 kb PCR product when oCTT36 and oCTT37 were used, indicating the presence of transformed chloroplast DNA. The two C-term MIS lines that yielded no PCR product could be due to poor transformation of those plants. As expected, genomic DNA from non-transformed plant did not yield a 1.8 kb PCR product and the control pLDpsbAHSA lines had a 1.8 kb PCR product. When the C-MIS gene specific primer oCTT14 and TpsbA 3′UTR primer oCTT18 were used the expected 0.5 kb PCR product was present in genomic DNA from C-term MIS lines that had yielded PCR products using oCTT36 and oCTT37. These results indicate that nine of the C-term MIS plant lines have the C-term MIS expression cassette integrated into the chloroplast genome. PCR analysis using primers oCTT36 and oCTT37 of the pLDrbs proMIS and pLDPpsbA proMIS transformed plants also showed the presence of transformed chloroplast genomes.

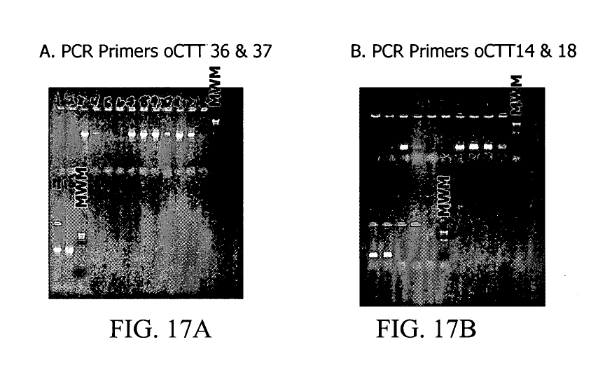

Seed from T₀ pLDrbsC-term MIS and pLDPpsbA C-term MIS plants were collected, sterilized and plated ˜200 seeds per plate on MS agar plates with 500 ug/mL spectinomycin. The seeds were germinated in a Conviron growth chamber with 12 hr light dark cycle at 28° C. Spectinomycin sensitive seedlings (non-transformed plants) will be bleached white when germinated and grown under these conditions while transformed seedlings will be green. rbs C-term MIS & PpsbA C-term MIS lines exhibited 100% spectinomycin resistant seedlings. PCR analysis then confirmed the presence of transformed chloroplasts with integrated rbs C-term MIS expression cassette. See FIG. 18 where transformed seedlings grown on spectinomycin selection are green.

C-term MIS from transplastomic C-term MIS plants can be extracted in sodium hydroxide. Five to 10 gm of tissue was homogenized in a blender at maximum speed for 5 min (Waring Blender) with 2 mL 50 mM NaOH per gm tissue. The homogenate was filtered through 2 layers of cheese cloth and 2 layers of Miracloth (CalBiochem, Inc.) to remove large tissue debris. The pH was adjusted to 12.0 with 10 N NaOH and the filtrate was spun at 10,000 rpm for 5 min to remove cell debris. Immunodetection of MIS was then performed on the samples. For example, western blot analysis of protein samples was performed using rabbit polyclonal antibody to CHO-derived C-term MIS and proMIS (gift of P Donahoe, Massachusetts General Hospital). Total soluble protein of the extract supernantant was determined using the Coomassie Plus Kit as directed by the manufacturer (Pierce, Inc). Twenty ug of total soluble protein was analyzed on Tris glycine 8-16% acrylamide gels (Mini-Protein® 3 Cell, BioRad, Inc) with 0.6 ug of control cleaved proMIS derived from CHO cells (gift of P Donahoe, MGH). The protein gel was transferred to 0.22 micron nitrocellulose filter (Pierce, Inc) as directed by manufacturer (Criterion™ Blotter, BioRad, Inc). The membrane was blocked overnight at room temperature in Tris-Casein (BioRad, Inc). Primary rabbit polyclonal diluted 1:1000 in Tris-Casein was used to treat the membrane for 1 hour at room temperature with gentle shaking. After treatment with primary antibody the membrane was washed in 0.2 M Tris pH 7.5, 0.005 M EDTA, and 0.1% (v/v) Tween 20 (TTEB). Alkaline phosphatase conjugated anti rabbit IgG (BioRad) diluted 1:3000 in Tris Casein was used to treat the membrane at room temperature with gentle shaking. After 45 min the membrane was washed in TTEB and treated with ImmunoSTAR and Enhance (BioRAD, Inc) for 5 min then exposed to X-ray film and developed. When protein extracts from rbsC-term MIS and PpsbAC-term MIS plants were analyzed under reducing denaturing conditions, cross-reacting 12 kDa protein that co-migrated with the CHO-derived cleaved MIS was detected.

Purification of MIS from the plant-derived material was done using salt precipitation of proteins followed by anion exchange chromatography using a DEAE column. This method exploits different properties of the MIS protein. The behavior of bioactive MIS on this column is known (D. MacLaughlin per communication). In addition to DEAE chromatography (Lorenzo, H K, Teixeira J, Pahlavan N, Laurich M V, Donahoe P K, MacLaughlin D T. Mullerian Inhibiting Substance: purification to homogeneity with increased yield and potency. J Chromatogr B Biomed Sci Appl. 2002 Jan. 5; 766(1):89-98), size fractionation and cation exchange chromatography was used according to manufacturer's recommendation (Amersham-Pharmacia). Detection of MIS protein was determined by western blot analysis of SDS-PAGE and slot blots using rabbit polyclonal antibodies to CHO-derived MIS.

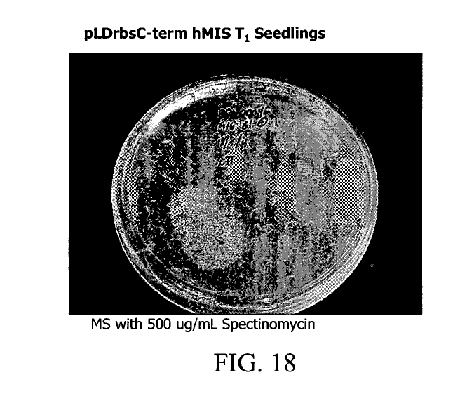

For example, leaves from transformed MIS plants were homogenized using a blender at maximum speed for 5 min with 2 mL cold 200 mM Tris pH 7.5, 5 mM EDTA, 0.1% (v/v) Tween-20 per gm tissue. The homogenate was filtered through 4 layers of cheese cloth and 2 layers of Miracloth (CalBioChem, Inc), adjusted to a pH of 4.5 using 5 N HCL, and tissue debris was pelleted at 10,000 g for 15 min. After collection of the supernatant, ammonium sulfate was used to partially precipitate proteins. For example at a final concentration of 40% (w/v). The proteins were salt precipitated for a minimum of two hours at 4 C. The protein precipitant was collected by centrifugation at 10,000 g for 15 min., solubilized in 25 mM glycine (pH 10.5) and either dialyzed in 25 mM glycine (pH 10.5) for 2 hrs at 4 C or passed through a desalting column (Amersham-Pharmacia, Inc) to remove the salt. The MIS protein was purified by anion exchange chromatography (MonoQ chromatography, Amersham-Pharmacia, Inc) as directed by the manufacturer. Protein samples were analyzed by western blot analysis of SDS-PAGE (FIG. 19). Slot-blot western immunodetection can also be used. For example, samples were diluted in 50 mM Tris pH 8.0, 0.3 M NaCl and 50 uL of each were loaded through 0.2 micron nitrocellulose membrane (Piece Inc) using a slot blot apparatus (EASY-TITER™, Pierce Inc.). CHO-derived cleaved MIS at 50 to 0.75 ng was used as a control. The membrane was dried at room temperature for 30 min prior to blocking in Tris-Casein (BioRad, Inc) and processing as described above.

When pro-MIS was extracted from rbs pro-MIS plant tissue and analyzed by western blot analysis, cross-reacting protein bands co-migrating with CHO-derived MIS were detected when rabbit polyclonal anti-CHO derive pro MIS was used. Pro MIS was detected in the crude TTEB homogenate and the 40% AS pellet samples (FIG. 19). This is an indication that pro-MIS from plants can be purified from transplastomic tissue.

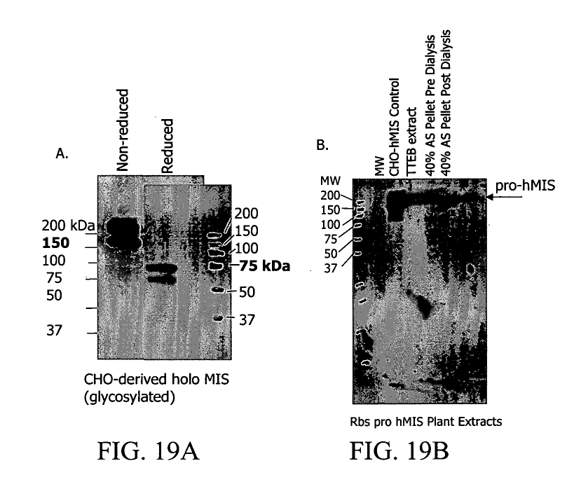

C-term MIS was extracted from PpsbA C-term MIS plant tissue, partially purified by salt precipitation and a 10 mL preparative anion (DEAE) exchange chromatography column, and analyzed by Dot-Blot immunoblot analysis (FIG. 20). C-term MIS was detected in the 40% ammonium sulfate precipitated protein fraction, the DEAE flow-through material and in the 100 mM NaCl elution fractions. Limited detection was found in the 300 mM NaCl elution fractions. This was an indication that C-term MIS from transplastomic tissue binds to DEAE and can be eluted in 100 mM NaCl.

When C-term MIS was extracted from PpsbA C-term MIS and rbs C-term MIS plant tissue, partially purified by salt precipitation of the cleared homogenate and analyzed by western blot analysis under non-reducing conditions, a 25 kDa cross-reacting protein band was detected in 30% ammonium sulfate precipitated pellet samples when rabbit polyclonal antibody to CHO-derived C-term MIS was used as the primary antibody.

### EXAMPLE 3

### Bioassays

Once the MIS protein is purified from transplastomic plant tissue, the protein can be assayed for bioactivity using, for example, the standard organ culture bioassay for MIS (MacLaughlin, D. T., Hudson, P. L., Graciano, A. L., Kenneally, M. K., Ragin, R. C., Manganaro, T. F. & Donahoe, P. K. (1992) “Mullerian duct regression and antiproliferative bioactivities of Mullerian Inhibiting Substance reside in its carboxy-terminal domain” Endocrinology 131, 291-296) can be used to screen all plant generated samples for bioactivity and the results compared to purified MIS secreted from CHO cells (Cate, R. L., Mattaliano, R. J., Hession, C., Tizard, R., Farber, N. M., Cheung, A., Ninfa, E. G., Frey, A. Z., Gash, D. J., Chow, E. P., Fisher, R. A., Bertonis, J. M., Torres, G., Wallner, B. P., Ramachandran, K. L., Ragin, R. C., Manganaro, T. F., MacLaughlin, D. T. & Donahoe, P. K. (1986). Isolation of the bovine and human genes for Mullerian Inhibiting Substance and expression of the human gene in animal cells. Cell 45, 685-698) and ion exchange chromatographic means. Briefly, 14½ day female fetal rat urogenital ridges are placed on agar-coated stainless steel grids above fortified CMRL 1066 media (GIBCO/BRL, Gaithersburg, Md.) containing 10% female fetal calf serum (to avoid contamination with bovine MIS) and the MIS containing sample to be assayed. All test materials and MIS standards will be tested in quadruplicate for each dose employed. After 3 days of incubation, specimens are fixed in 15% formalin, embedded in paraffin, cut in 8 mm serial sections, stained, and scored from grade 0 (no regression) to grade 5 (complete regression) by two experienced observers. One unit of activity is defined as causing a 1 grade increase in Mullerian duct regression (MacLaughlin et al., 1991). Dose responses for either the cleaved holo- or C-terminal MIS standard proteins and the plant generated MIS fragments can be done so that the ED50 for Mullerian duct regression can be compared for all proteins.

### EXAMPLE 4

### Generation of Tobacco Biomass

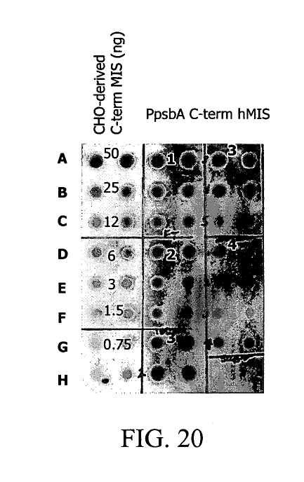

Seeds from transplastomic rbs C-term MIS and PpsbA C-term MIS were propagated in the greenhouse and field in soil with no spectinomycin selection (FIG. 21). When genomic DNA was analyzed by PCR analysis, plant tissue was found to contain the C-term MIS expression cassette in the chloroplast genome. When the total protein was extracted and purified, the C-term MIS was detected in the protein extracts using western blot analysis as described above. This is an indication that the transplastomic plants grown under greenhouse and field conditions maintain the transformed chloroplast with the functional expression cassette.

### EXAMPLE 5

### His-tagged HoloMIS

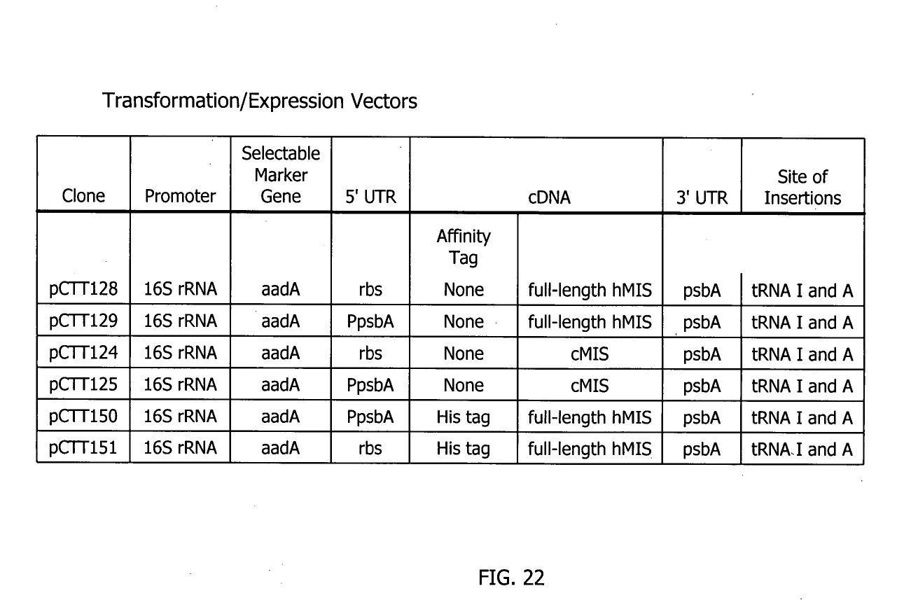

Expression cassettes were generated producing non-native hMIS coding sequences. To enhance the efficiency of purification of hMIS, a 6-His amino tag has been linked to the N-terminal of hMIS (see FIG. 23) The full-length HMIS was designed to contain an N-terminal affinity tag, His-tag. As shown in FIG. 23, the bold letters in panel A represent the nucleotide sequence of the His-tag and the bold letters in panel B represent the amino acid sequence of the His-tag. Oligonucleotide 5′ primers were designed containing the SphI site, His-tag sequence (FIG. 23) and N-terminal HMIS sequence. The 3′ primer used in Example 1 was used to PCR amplify the HisTag-hMIS cDNA. The SphI/XbaI digested PCR product was cloned into the SphI/XbaI digested pLD vector used previously (see Example 1) to generate the pCTT150. The rbs Histag HMIS clone was generated using a similar method. The designed 5′primer contained the SphI site with rbs sequence and N-terminal HMIS sequence. This primer was used to generate the 5′rbs UTR expression vector pCTT151. Both pCTT150 and pCTT151 were used to generate transplatomic plants expressing a His-tagged hMIS fusion protein.

### EXAMPLE 6

### Purification of hMIS

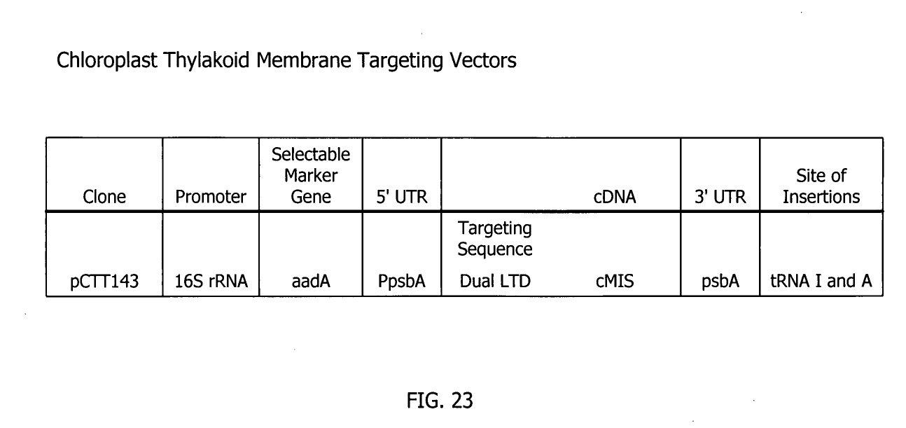

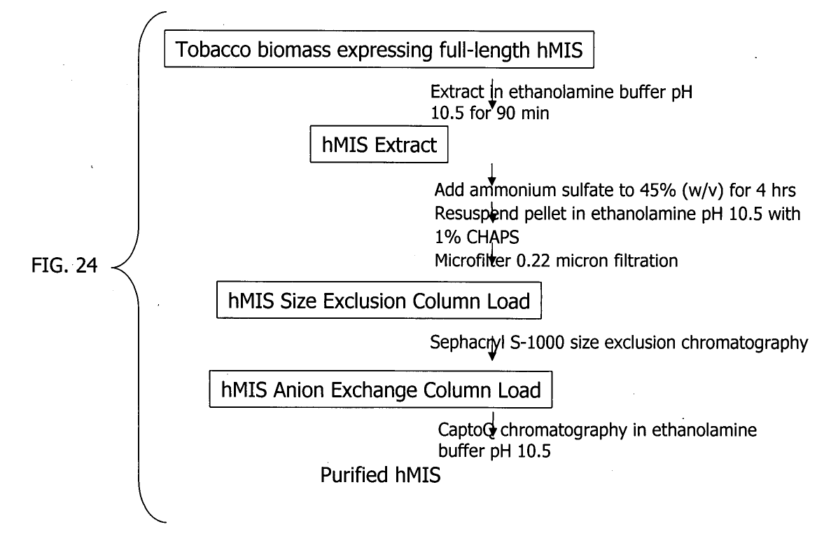

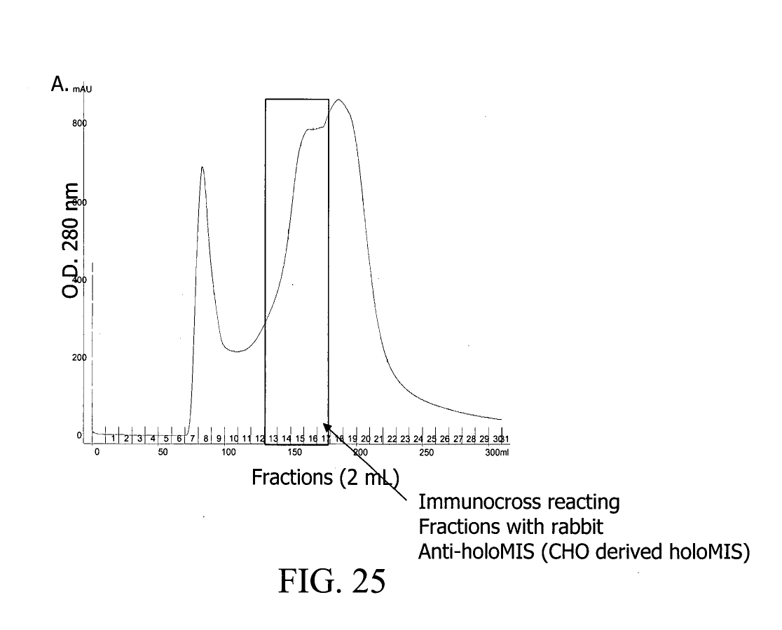

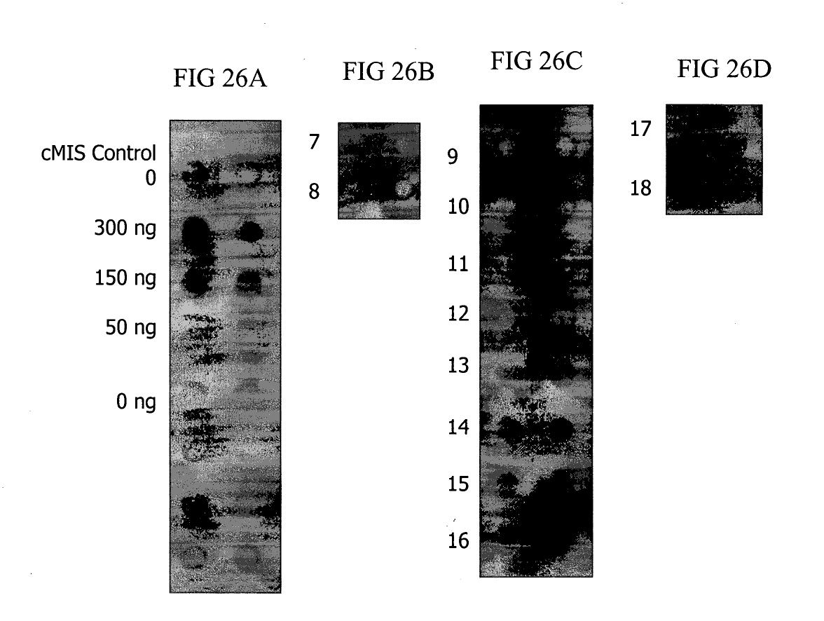

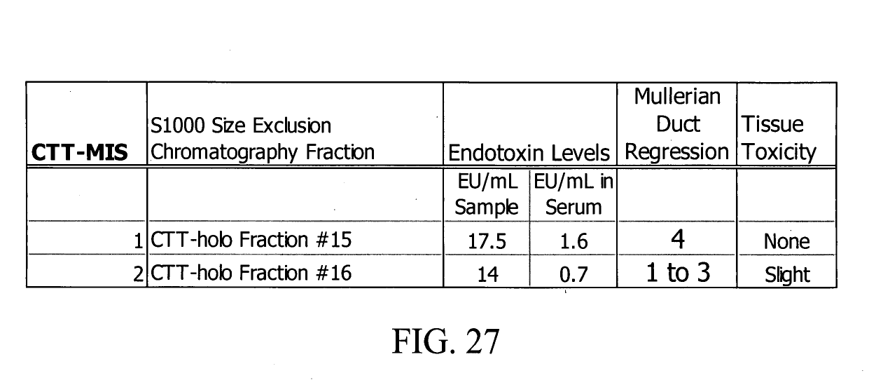

Leaves from transformed hMIS tobacco plants (pCTT128 vector) were processed through a Greenstar twin screw press or homogenized using a blender. The resulting liquid plant extract was placed into ethanolamine buffer with a final concentration of 100 mM ethanolamine pH 10.5, 5 mM EDTA, 0.01% (v/v) Tween-20, 0.1% (w/v) metabisulfite and 1 mM benzamidine. The ratio of biomass or plant exudates to buffer was 1:2. The pH of the homogenate was adjusted to 10.5 with sodium hydroxide. The debris was pelleted by centrifugation at 8,000 g at 4° C. for 30 min. Using standard procedures for the purification of proteins (Deutscher, M P, Guide to Protein Purification: Methods in Enzymology, Academic Press), hMIS was purified (FIG. 24). The proteins in the clarified homogenate were precipitated with ammonium sulfate at a final concentration of 45% (w/v) for 4 hrs (Deutscher, M P, Guide to Protein Purification: Methods in Enzymology, Academic Press). The ammonium sulfate pellet was collected by centrifugation at 12,000 g for 30 min at 4° C. followed by suspension in the ethanolamine buffer containing plant protease inhibitor cocktail (Sigma Aldrich) and 1% (w/v) CHAPS. The protein extract was microfiltered through a 0.2 micromembrane to remove insoluble debris. The resulting filtrate was loaded onto a Sephacryl S1000 size exclusion column. Protein fractions were assayed for protein content, MISRII-Fc and cross reactivity to rabbit anti-holoMIS binding. Protein fractions with the strongest MISRII-Fc binding activity were dialyzed in PBS pH 7.5 (10 mM sodium phosphate, 150 mM sodium chloride pH 7.5) overnight prior to being tested for MIS bioactivity. As shown in FIG. 27, fractions 15 and 16 had significant Mullerian Duct regression activity. This shows that full-length hMIS expressed and purified from plant tissue has been assembled as a homodimer and cleaved to produce bioactive cMIS. The hMIS can be further purified by several standard methods. hMIS protein resulting from size exclusion chromatography was diafiltered in ethanolamine buffer pH 10.5 and loaded onto anion exchange resin and eluted in a 0 to 1 M NaCl gradient in ethanolamine buffer (FIG. 24). As shown in FIG. 28, 11.7 kDa cMIS protein was detected by western blot analysis using a rabbit polyclonal anti-hMIS antibody in samples eluted from an anion exchange column. Chloroplast derived cross-reacting protein was found to co-migragte with E. coli derived cMIS control protein (RDI systems). This is a confirmation that chloroplast expressed and purified HMIS can be assembled and cleaved.

### EXAMPLE 7

### Purification of cMIS

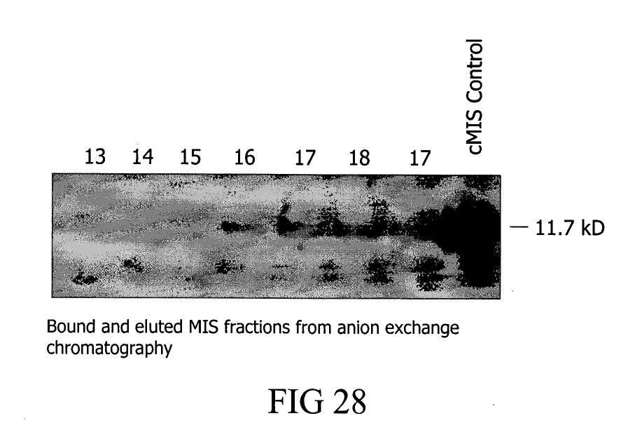

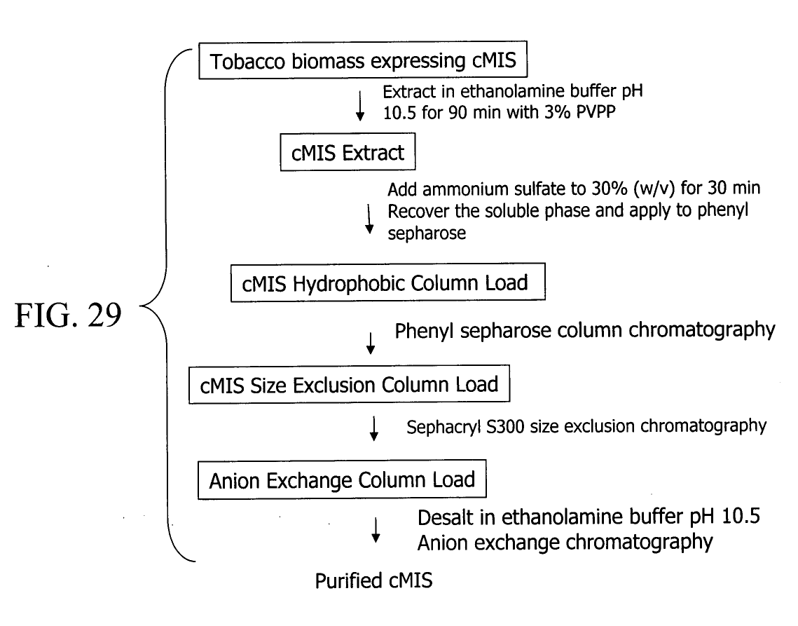

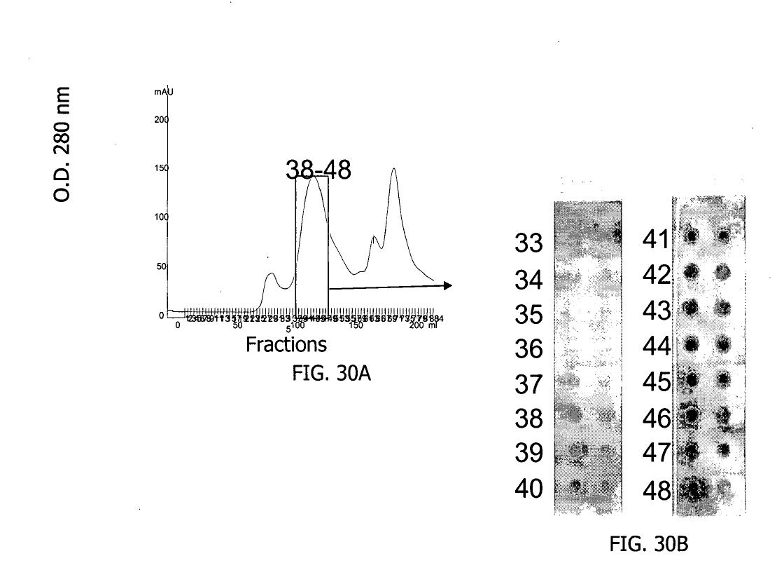

Leaves from transformed cMIS tobacco plants (pCTT125 vector) were processed through a Greenstar twin screw press or homogenized using a blender. The resulting liquid plant extract was placed into ethanolamine buffer with a final concentration of 100 mM ethanolamine pH 10.5, 5 mM EDTA, 0.01% (v/v) Tween-20, 0.1% (w/v) metabisulfite and 1 mM benzamidine. The ratio of biomass or plant exudates to buffer is 1:2. The pH of the homogenate was adjusted to 10.5 with sodium hydroxide. The debris was pelleted by centrifugation at 8,000 g at 4° C. for 30 min. Using standard procedures for the purification of proteins (Deutscher, M P, Guide to Protein Purification: Methods in Enzymology, Academic Press), cMIS was purified (FIG. 29). Endogenous proteins were removed by ammonium sulfate precipitation at 30% (w/v). Endogenous proteins were removed by centrifugation at 20,000 g for 30 min at 4° C. The soluble fraction was passed through a 0.22 micron filter to remove all insoluble debris, loaded onto phenyl sepharose column chromatography and eluted in 1 to 0 M ammonium sulfate linear gradient. Samples from fractions were analyzed by slot blot ELISA using chicken anti-cMIS antibodies. The cMIS positive fractions were pooled and fractionated by sepharcryl S300 size exclusion column chromatography. See FIG. 30, panel A. Samples from each fraction were analyzed by slot blot elisa with MISRII-Fc to identify fractions with MISRII-Fc binding activity. See FIG. 30, panel B. As shown in FIG. 30, fractions 38-48 had strong MISRII-Fc binding activity. These fractions were pooled and loaded onto Resource Q anion exchange column chromatography (FIG. 31) and eluted with 0-1 M sodium chloride linear gradient (FIG. 31, panel A). Samples from each fraction were analyzed by slot blot elisa using MISRII-Fc (FIG. 31, panel B). As shown in FIG. 31, panel B, fractions 13 and 14 had the strongest MISRII-Fc binding activity. This shows that there was an enrichment for MISRII-Fc binding activity in these protein fractions.

### EXAMPLE 8

### Soluble MIS Receptor RII-Fc (MISRII-Fc) and ELISA

Similar to all members of the TGF-beta family of proteins, hMIS signals through two serine protease kinases. The bioactive carboxyl-terminal fragments of these cell-cycle regulators bind as dimers to heteromeric complex of serine/threonine kinase transmembrane receptors, type I and type II. Type II MIS receptor (Barrends, W M, et al (1994) Development 120:189-197; Mishina et al. (1997) Biochem and Biophys Res Comm 237:741-746.) genes have been isolated and characterized (Mishina et al 1997, Biochem Biophys Res Commun 237:741-746). The MISRII is found in mesenchylmal cells which are adjacent to the Mullerian duct epithelium, in Sertoli and granulose cells (Teixeria et al 1996, Endocrinology 137:160-165; Baarends et al 1994, Development 120:189-137; diClemente et al. 1994, Mol Endocrinol 8:1006-1020). Similar to other TGF-beta family transmembrane receptor proteins, the MIS type II receptor protein (MISRII) has a single binding N terminal domain that can be cloned and expressed separately from the transmembrane domain as a soluble receptor (Derynck and Feng 1997; del Re et al., 2004). Similar to other soluble receptors which have been used as diagnostic or potential therapeutic drugs (Maxwell et al. 1999, Wooley et al. 1993; Haraoui et al. (2000), the soluble receptor domain of MISRII has been fused to Fc domain of a human IgG antibody. The soluble MISRII-Fc fusion protein has been used to detect carboxyl-terminal dimer of MIS in Elisa. The MISRII-Fc protein is a dimer with two MISRII binding sites per assembled protein. MISRII cDNA has been isolated from human, mice, rat and rabbit. Soluble MISRII-Fc recombinant proteins from R&D Systems (cat#1618MR) and Drs. Donahoe and MacLaughlin at Mass. General Hospital (Boston, Mass.) which has been demonstrated to have specificity for the bioactive cMIS fragment.

One to two ug of total soluble protein from purification steps or control protein samples were bound to a 0.22 micron nitrocellulose membrane by dot blotting methods as decribed previously. The resulting membranes were rinsed in TBST (20 mM Tris pH 7.5, 500 mM NaCl, 0.1% Tween-20) for 15 min at room temperature. The membrane was placed into a glass hybridization bottle with TBST and 5% (w/v) non-fat dried milk at 30° C. and slow rotation. After 1 hr the membrane was placed in TBST and 5% non-fat dried milk containing 1.4 ug/mL MISRII-Fc soluble receptor protein with slow rotation at 30° C. After 16 hr the membrane was removed from the hydridization bottle and washed in TBST for 15 min with agitation. The membrane was placed into TBST and 5% non-fat dried milk containing 1:15,000 dilution of secondary antibody, goat anti-human IgG Fc alkaline phosphatase conjugated (Sigma A8438), at room temperature. After 1 hr the membrane was washed in TBST for 15 min, rinsed with chemo-luminescent substrate (BioRAD) for 5 min and exposed to x-ray film. The intensity of the exposed film is an indication of the level of cMIS present in each sample.

We claim:

1. A transgenic plastid which comprises a plastid genome that contains a heterologous polynucleotide sequence that encodes a full length or truncated TGF-β protein wherein the TGF-β protein is expressed in the transgenic plastid and the TGF-β protein is bioactive.

2. The transgenic plastid of claim 1 wherein the TGF-β protein is Holo-MIS or C-Term MIS.

3. The transgenic plastid of claim 2 wherein the transgenic plastid is a chloroplast.

4. The transgenic plastid of claim 3 wherein the chloroplast is a tobacco chloroplast.

5. A pharmaceutical formulation which comprises (a) a full length or truncated TGF-β protein that was made in a transgenic plant plastid and (b) a pharmaceutically acceptable carrier.

6. The pharmaceutical formulation of claim 5 wherein the TGF-β protein is Holo-MIS or C-Term MIS.

7. The pharmaceutical formulation of claim 6 wherein the transgenic plastid is a chloroplast.

8. The pharmaceutical formulation of claim 7 wherein the chloroplast is a tobacco chloroplast.

9. A method of binding a protein to a TGF-β receptor in a mammal which comprises administering to the mammal an effective amount of a full length or truncated TGF-β protein that has been made in a plant plastid wherein the full length or truncated TGF-β protein binds to its corresponding TGF-β receptor.

10. The method of claim 9 wherein the TGF-β protein is Holo-MIS or C-Term MIS.

11. The method of claim 10 wherein the transgenic plastid is a chloroplast.

12. The method of claim 11 wherein the chloroplast is a tobacco chloroplast.

13. A method of treating a patient having ovarian cancer that contains MIS receptors which comprises administering to the patient an effective MIS-receptor binding amount of a full length or truncated MIS protein wherein (a) the full length or truncated MIS protein was made in a plant plastid and (b) the full length or truncated MIS protein binds with the MIS receptors present in the ovarian cancer.

14. The method of claim 13 wherein the TGF-β protein is Holo-MIS or C-Term MIS.

15. The method of claim 14 wherein the transgenic plastid is a chloroplast.

16. The method of claim 3 wherein the chloroplast is a tobacco chloroplast.

17. A plant cell comprising a plastid including a DNA construct comprising, as operably joined components, (a) one or more regulatory sequences functional in said plastid, (b) a heterologous DNA sequence encoding a full length or truncated TGF-β protein, and (c) a second heterologous DNA sequence encoding a selectable marker protein, wherein transcription of said DNA sequences is regulated by said one or more regulatory sequences.

18. The plant cell of claim 17 wherein the TGF-β protein is Holo-MIS or C-Term MIS.

19. The plant cell of claim 18 wherein the transgenic plastid is a chloroplast.

20. The plant cell of claim 19 wherein the chloroplast is a tobacco chloroplast.

21. The plant cell of claim 20 wherein the regulatory sequences include (a) a promoter or a 5′ UTR upstream of the heterologous DNA sequences and (b) a 3′ termination sequence downstream from the heterologous DNA sequences.

22. The plant cell of claim 21 wherein the selectable marker protein inactivates a selection agent that is toxic to the tobacco chloroplasts but not to the tobacco plant cell.

23. A stably transformed transcription/translation active plastid of a higher plant, which is competent for uptake of exogenous DNA, which comprises an expression cassette comprising (a) exogenous DNA comprising a coding sequence for a full length or truncated TGF-β protein and (b) one or more regulatory sequences functional in plastids wherein the DNA is stably integrated and inherited through organelle replication in daughter cells.

24. The plastid of claim 23 wherein the TGF-β protein is Holo-MIS or C-Term MIS.

25. The plastid of claim 24 wherein the transgenic plastid is a chloroplast.

26. The plastid of claim 25 wherein the chloroplast is a tobacco chloroplast.

27. The plastid of claim 26 wherein the regulatory sequences comprise (a) a promoter or a 5′ UTR upstream of the heterologous DNA sequences and (b) a 3′ termination sequence downstream from the heterologous DNA sequences.

28. An expression cassette for stably transforming plastids of higher plants comprising (a) heterologous DNA comprising a coding sequence for a full length or truncated TGF-β protein and (b) one or more regulatory sequences functional in plastids.

29. The expression cassette of claim 28 wherein the TGF-β protein is Holo-MIS or C-Term MIS.

30. The expression cassette of claim 29 wherein the plastid is a chloroplast.

31. The expression cassette of claim 30 wherein the chloroplast is a tobacco chloroplast.

32. The expression cassette of claim 31 wherein the regulatory sequences comprise:
(a) a promoter or a 5′ UTR upstream of the heterologous DNA sequence and
(b) a 3′ termination sequence downstream from the heterologous DNA sequence.

33. A stable plastid transformation and expression vector competent for stably transforming a plastid genome which comprises:
(a) an expression cassette comprising as operably linked components
(i) one or more regulatory sequences functional in said plastid,
(ii) a selectable marker coding sequence,
(iii) a heterologous DNA sequence coding for a full length or truncated TGF-β protein and
(b) flanking each side of the expression cassette, flanking DNA sequences which are homologous to a DNA sequence inclusive of a spacer sequence of the target plastid genome, whereby stable integration of the expression cassette into the plastid genome of the target plant is facilitated through homologous recombination of the flanking sequences with the homologous sequences in the target plastid genome.

34. The vector of claim 33 wherein the TGF-β protein is Holo-MIS or C-Term MIS.

35. The vector of claim 34 wherein the transgenic plastid is a chloroplast.

36. The vector of claim 35 wherein the chloroplast is a tobacco chloroplast.

37. The vector of claim 36 wherein the regulatory sequences include:
(a) a promoter or a 5′ UTR upstream of the heterologous DNA sequence and the selectable marker coding sequence and
(b) a 3′ termination sequence downstream from the heterologous DNA sequence and the selectable marker coding sequence.

38. The vector of claim 37 wherein the selectable marker sequence encodes a protein that inactivates a selection agent that is toxic to the tobacco chloroplasts but not to the tobacco plant cell.

39. A plastid transformation vector for stably transforming a plastid, said plastid vector comprising:
(a) a first flanking sequence,
(b) a DNA sequence containing
(i) a coding region for a TGF-β protein and
(ii) one or more regulatory sequences functional in said plastid, and
(c) a second flanking sequence wherein both of said flanking sequences are homologous to a DNA sequence of the target plastid genome wherein the flanking sequences facilitate the stable integration of the DNA sequence into the plastid genome through homologous recombination.

40. The vector of claim 39 wherein the TGF-β protein is Holo-MIS or C-Term MIS.

41. The vector of claim 40 wherein the transgenic plastid is a chloroplast.

42. The vector of claim 41 wherein the chloroplast is a tobacco chloroplast.

43. The vector of claim 42 wherein the regulatory sequences include (a) a promoter or a 5′ UTR upstream of the TGF-β coding region and downstream to the first flanking sequence and (b) a 3′ termination sequence downstream from the TGF-β coding region and upstream to the second flanking sequence.

44. A method of producing a biologically active TGF-β protein which comprises expressing a coding sequence for a TGF-β protein or a fragment thereof in a plastid of a plant.

45. The method of claim 44 wherein the TGF-β protein is a full length protein which is processed in the plant tissue into a biologically fragment thereof.

46. The method of claim 45 wherein the TGF-β protein is full length MIS and the full length MIS is cleaved when purifying said full length MIS from plant biomass into C-terminal MIS.

47. An aglycosylated Mullerian Inhibitor Substance (MIS) protein.

48. The aglycosylated MIS protein of claim 47 which is a human MIS monomer having a molecular weight of about 55 kDa.

49. The aglycosylated MIS protein of claim 47 which is a human MIS dimer having a molecular weight of about 110 kDa.

50. In a method of making human Mullerian Inhibitor Substance (MIS) protein by (i) growing a transgenic plant with an expression cassette containing a coding region for human MIS to form a plant biomass and (ii) purifying the human MIS from the plant biomass, the improvement which comprises:
employing a transplastomic plant having said coding region in the plastid whereby the expressed human MIS protein is bioactive.


SEQUENCE LISTING (21 sequences)

SEQ ID NO: 1 - DNA, 30 bp, Artificial Sequence
  oCTT1 primer
ggaggcaaccatgtccgctggtgctactgc

SEQ ID NO: 2 - DNA, 28 bp, Artificial Sequence
  oCTT2 primer
gaattcggcttggaggcaaccatgtccg

SEQ ID NO: 3 - DNA, 194 bp, Artificial Sequence
  Chloroblast psbA promoter (PpsbA) flanked with an EcoRI restriction site
gaattcgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttacc

SEQ ID NO: 4 - protein, 110 aa, Artificial Sequence
  Amino acid sequence of carboxyl terminal bioactive human Mullerian Inhibiting Substance (C-term hMIS) for CTT expression
MSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 5 - DNA, 336 bp, Artificial Sequence
  Nucleotide sequence of the coding sequence of carboxyl terminal hMIS synthetically produced for generation of CTT transformation expression cassettes
atgtccgctggtgctactgctgcagatggtccttgtgctcttcgtgaactttctgtagacttaagagctgaaagatctgtactaattcctgaaacttatcaagctaacaattgtcaaggtgtatgtggttggcctcaatctgatcgtaatcctcgttatggaaatcatgttgttctattattaaaaatgcaagctcgcggcgcagctcttgcaagacctccatgttgtgttcctactgcttatgctggtaaactattaatctccctatctgaagagcgtatctctgctcaccatgttcctaatatggttgctaccgaatgtggttgccgttaataa

SEQ ID NO: 6 - DNA, 536 bp, Artificial Sequence
  536 nt EcoRI/XbaI 5′PpsbA C-term hMIS
gaattcgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttaccatgtccgctggtgctactgctgcagatggtccttgtgctcttcgtgaactttctgtagacttaagagctgaaagatctgtactaattcctgaaacttatcaagctaacaattgtcaaggtgtatgtggttggcctcaatctgatcgtaatcctcgttatggaaatcatgttgttctattattaaaaatgcaagctcgcggcgcagctcttgcaagacctccatgttgtgttcctactgcttatgctggtaaactattaatctccctatctgaagagcgtatctctgctcaccatgttcctaatatggttgctaccgaatgtggttgccgttaataatctaga

SEQ ID NO: 7 - DNA, 363 bp, Artificial Sequence
  rbsC-term hMIS Expression Cassette
gaattcggcttggaggcaaccatgtccgctggtgctactgctgcagatggtccttgtgctcttcgtgaactttctgtagacttaagagctgaaagatctgtactaattcctgaaacttatcaagctaacaattgtcaaggtgtatgtggttggcctcaatctgatcgtaatcctcgttatggaaatcatgttgttctattattaaaaatgcaagctcgcggcgcagctcttgcaagacctccatgttgtgttcctactgcttatgctggtaaactattaatctccctatctgaagagcgtatctctgctcaccatgttcctaatatggttgctaccgaatgtggttgccgttaataatctaga

SEQ ID NO: 8 - protein, 428 aa, Artificial Sequence
  N-terminal hMIS
MRAEEPAVGTSGLIFREDLDWPPGIPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRS

SEQ ID NO: 9 - DNA, 1284 bp, Artificial Sequence
  N-terminal hMIS
atgcgagctgaagaacctgcagtaggcacttctggtttaatttttcgtgaagacctagattggcctcctggtatccctcaagaacctctttgtcttgttgctttaggtggtgattccaacggttctagctctcctctacgtgttgttggtgctttatctgcatacgaacaggcttttcttggtgcagtacaacgtgctcgttggggtcctcgtgatctagctactttcggtgtttgtaatactggtgatcgtcaagctgctctaccttctcttcgtcgtcttggtgcttggcttcgtgatcctggtggtcaacgtttagtagttttacaccttgaagaagtaacctgggaacctactccatccttacgtttccaagaacctccacctggtggcgctggtcctcctgaattggctttacttgttttatatcctggtcctggtcctgaagttaccgttactcgtgctggtcttcctggtgctcaatctctttgtccttcccgtgatacccgttatttagttcttgctgttgatcgtcctgctggtgcttggcgtggatctggtcttgctcttactttacaacctcgtggtgaagattctcgtttatccactgctcgtttacaagctctactttttggtgatgatcaccgttgtttcactcgtatgactcctgctcttcttcttttacctcgttccgaacctgctcctcttcctgctcatggtcaacttgatactgttcctttccctcctcctcgtccttccgctgaattagaagaatctcctccatctgctgatcctttcctagaaactctaactcgtcttgttcgtgctcttcgtgttcctcctgctcgtgcttctgctcctcgcttagctcttgatcctgatgctctagctggtttccctcaaggtctagttaatttatccgatcctgctgctttagaacgtttattagacggtgaagaacctctattattgctactacgtccaactgctgctactactggtgatcctgctcctttacatgaccctacctctgctccttgggctactgctcttgctagacgtgttgctgctgaactacaagctgctgctgctgaacttcgctctctacctggtttacctcctgcaaccgctccattgttggctcgtcttttagctctatgtcctggtggtcctggtggtcttggtgatccattacgcgctctattactattaaaagctctacaaggtttacgtgttgaatggcgtggacgtgatcctcgtggtcctggccgtgcacaacgatcg

SEQ ID NO: 10 - DNA, 40 bp, Artificial Sequence
  oCTT26 primer
aagaattcggcttggaggcaaccatgcgagctgaagaacc

SEQ ID NO: 11 - DNA, 29 bp, Artificial Sequence
  oCTT27 primer
ctagtctagacgatcgttgtgcacggcca

SEQ ID NO: 12 - DNA, 25 bp, Artificial Sequence
  oCTT13 primer
atcgatcggctggtgctactgctgc

SEQ ID NO: 13 - DNA, 1641 bp, Artificial Sequence
  1641 nt EcoRI, rbs--synthetic proMIS, XbaI
gaattcggcttggaggcaaccatgcgagctgaagaacctgcagtaggcacttctggtttaatttttcgtgaagacctagattggcctcctggtatccctcaagaacctctttgtcttgttgctttaggtggtgattccaacggttctagctctcctctacgtgttgttggtgctttatctgcatacgaacaggcttttcttggtgcagtacaacgtgctcgttggggtcctcgtgatctagctactttcggtgtttgtaatactggtgatcgtcaagctgctctaccttctcttcgtcgtcttggtgcttggcttcgtgatcctggtggtcaacgtttagtagttttacaccttgaagaagtaacctgggaacctactccatccttacgtttccaagaacctccacctggtggcgctggtcctcctgaattggctttacttgttttatatcctggtcctggtcctgaagttaccgttactcgtgctggtcttcctggtgctcaatctctttgtccttcccgtgatacccgttatttagttcttgctgttgatcgtcctgctggtgcttggcgtggatctggtcttgctcttactttacaacctcgtggtgaagattctcgtttatccactgctcgtttacaagctctactttttggtgatgatcaccgttgtttcactcgtatgactcctgctcttcttcttttacctcgttccgaacctgctcctcttcctgctcatggtcaacttgatactgttcctttccctcctcctcgtccttccgctgaattagaagaatctcctccatctgctgatcctttcctagaaactctaactcgtcttgttcgtgctcttcgtgttcctcctgctcgtgcttctgctcctcgcttagctcttgatcctgatgctctagctggtttccctcaaggtctagttaatttatccgatcctgctgctttagaacgtttattagacggtgaagaacctctattattgctactacgtccaactgctgctactactggtgatcctgctcctttacatgaccctacctctgctccttgggctactgctcttgctagacgtgttgctgctgaactacaagctgctgctgctgaacttcgctctctacctggtttacctcctgcaaccgctccattgttggctcgtcttttagctctatgtcctggtggtcctggtggtcttggtgatccattacgcgctctattactattaaaagctctacaaggtttacgtgttgaatggcgtggacgtgatcctcgtggtcctggccgtgcacaacgatcggctggtgctactgctgcagatggtccttgtgctcttcgtgaactttctgtagacttaagagctgaaagatctgtactaattcctgaaacttatcaagctaacaattgtcaaggtgtatgtggttggcctcaatctgatcgtaatcctcgttatggaaatcatgttgttctattattaaaaatgcaagctcgcggcgcagctcttgcaagacctccatgttgtgttcctactgcttatgctggtaaactattaatctccctatctgaagagcgtatctctgctcaccatgttcctaatatggttgctaccgaatgtggttgccgttaataatctaga

SEQ ID NO: 14 - DNA, 28 bp, Artificial Sequence
  oCTT15 primer
cggaattcgtagagaagtccgtattttt

SEQ ID NO: 15 - DNA, 28 bp, Artificial Sequence
  oCTT21 primer
acatgcatgcgagctgaagaacctgcag

SEQ ID NO: 16 - DNA, 28 bp, Artificial Sequence
  oCTT22 primer
atgtgcatgctaaaatcttggtttattt

SEQ ID NO: 17 - DNA, 1814 bp, Artificial Sequence
  1814 nt EcoRI-XbaI PpsbA prohMIS
gaattcgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttagcatgcgagctgaagaacctgcagtaggcacttctggtttaatttttcgtgaagacctagattggcctcctggtatccctcaagaacctctttgtcttgttgctttaggtggtgattccaacggttctagctctcctctacgtgttgttggtgctttatctgcatacgaacaggcttttcttggtgcagtacaacgtgctcgttggggtcctcgtgatctagctactttcggtgtttgtaatactggtgatcgtcaagctgctctaccttctcttcgtcgtcttggtgcttggcttcgtgatcctggtggtcaacgtttagtagttttacaccttgaagaagtaacctgggaacctactccatccttacgtttccaagaacctccacctggtggcgctggtcctcctgaattggctttacttgttttatatcctggtcctggtcctgaagttaccgttactcgtgctggtcttcctggtgctcaatctctttgtccttcccgtgatacccgttatttagttcttgctgttgatcgtcctgctggtgcttggcgtggatctggtcttgctcttactttacaacctcgtggtgaagattctcgtttatccactgctcgtttacaagctctactttttggtgatgatcaccgttgtttcactcgtatgactcctgctcttcttcttttacctcgttccgaacctgctcctcttcctgctcatggtcaacttgatactgttcctttccctcctcctcgtccttccgctgaattagaagaatctcctccatctgctgatcctttcctagaaactctaactcgtcttgttcgtgctcttcgtgttcctcctgctcgtgcttctgctcctcgcttagctcttgatcctgatgctctagctggtttccctcaaggtctagttaatttatccgatcctgctgctttagaacgtttattagacggtgaagaacctctattattgctactacgtccaactgctgctactactggtgatcctgctcctttacatgaccctacctctgctccttgggctactgctcttgctagacgtgttgctgctgaactacaagctgctgctgctgaacttcgctctctacctggtttacctcctgcaaccgctccattgttggctcgtcttttagctctatgtcctggtggtcctggtggtcttggtgatccattacgcgctctattactattaaaagctctacaaggtttacgtgttgaatggcgtggacgtgatcctcgtggtcctggccgtgcacaacgatcggctggtgctactgctgcagatggtccttgtgctcttcgtgaactttctgtagacttaagagctgaaagatctgtactaattcctgaaacttatcaagctaacaattgtcaaggtgtatgtggttggcctcaatctgatcgtaatcctcgttatggaaatcatgttgttctattattaaaaatgcaagctcgcggcgcagctcttgcaagacctccatgttgtgttcctactgcttatgctggtaaactattaatctccctatctgaagagcgtatctctgctcaccatgttcctaatatggttgctaccgaatgtggttgccgttaataatctaga

SEQ ID NO: 18 - DNA, 25 bp, Artificial Sequence
  oCTT36 primer
aaaacccgtcctcagttcggattgc

SEQ ID NO: 19 - DNA, 25 bp, Artificial Sequence
  oCTT37 primer
ccgcgttgtttcatcaagccttacg

SEQ ID NO: 20 - DNA, 26 bp, Artificial Sequence
  oCTT14 primer
ccaagcttgtccgctggtgctactgc

SEQ ID NO: 21 - DNA, 28 bp, Artificial Sequence
  oCTT18 primer
caccctcttgatagaacaagaaaatgat